(19) United States
(12) Patent Application Publication
(10) Pub. No.: US 2024/0358745 A1
(43) Pub. Date: Oct. 31, 2024

(54) COMPOSITIONS AND METHODS OF DISSOLVING BIOFILM TO PROMOTE WOUND HEALING

(71) Applicant: Darlene E. McCord, Chattanooga, TN (US)

(72) Inventors: Darlene E. McCord, Chattanooga, TN (US); Alex Hovey, North Liberty, IA (US)

(21) Appl. No.: 18/649,065

(22) Filed: Apr. 29, 2024

Related U.S. Application Data
(60) Provisional application No. 63/498,625, filed on Apr. 27, 2023.
(60) Provisional application No. 63/512,756, filed on Jul. 10, 2023.

(51) Int. Cl. A61K 33/06; A61K 9/00; A61K 45/06; A61K 47/10; A61K 47/22; A61K 47/34; A61P 31/04

(52) U.S. Cl. CPC A61K 33/06; A61K 45/06; A61K 47/10; A61K 47/22; A61K 47/34; A61P 31/04; A61K 9/0014

(57) ABSTRACT

Antimicrobial compositions comprising at least one nonionic block EO-PO copolymer, at least one preservative system and/or medicinal clay, and polyacrylamide are provided. Associated methods of use and treatment are also provided, including methods of disrupting biofilms caused by the release of autoinducers, virulence factors and complicated by adhesion molecules and pathogen resistance, methods of treating chronic wounds, methods of inhibiting quorum sensing in a population of bacteria, and methods of interfering with biofilm cation signaling.

### CROSS REFERENCE TO RELATED APPLICATIONS

This application claims priority under 35 U.S.C. § 119 to provisional patent applications U.S. Ser. No. 63/498,625, filed Apr. 27, 2023, and U.S. Ser. No. 63/512,756, filed Jul. 10, 2023. The provisional patent applications are herein incorporated by reference in their entirety, including without limitation, the specification, claims, and abstract, as well as any figures, tables, appendices, or drawings thereof.

### TECHNICAL FIELD

The present disclosure relates generally to compositions and methods for the treatment and prevention of biofilms including pathogen removal.

### BACKGROUND

Biofilms are structured communities of microorganisms that are embedded in a self-produced matrix and form on various surfaces. Biofilms are complex surface attached communities of microbes held together by self-produced polymer matrices mainly composed of polysaccharides, secreted proteins, and extracellular DNAs. It is estimated that about 40-80% of bacterial cells on earth can form biofilms, including those commonly associated with infection and chronic wounds, for example, Staphylococcus aureus, Staphylococcus epidermidis, and Pseudomonas aeruginosa. Biofilms have a detrimental effect in healthcare settings and have been shown to develop on medical device surfaces, dead tissues, and inside living tissues. Biofilms pose a barrier to effective wound care by impairing wound healing and increasing the risk for wound infection.

Current treatment options for removing biofilms and curing chronic wounds are undesirable for various reasons. Regular debridement is a commonly prescribed treatment, but is painful, damaging to healthy tissues, and requires frequent clinical visits. Antibiotics are routinely prescribed to treat the underlying bacterial infection; however, many bacterial biofilms are resistant to antibiotics. Traditional biofilm treatments are often inadequate, causing patients to suffer from chronic wounds for years before resolution or worsening.

Thus, there is a need in the art for antimicrobial compositions that restrict the activity of autoinducers and adhesion molecules that are gentle and effective at removing and/or inhibiting microbial biofilms and treating chronic wounds.

It is an objective of the disclosure to provide antimicrobial compositions that overcome the limitations in the art.

It is another objective of this disclosure to formulate compositions for use in treating a subject, including providing wound healing.

It is another objective of this disclosure to formulate compositions for use in inhibiting quorum sensing in a population of bacteria by reducing autoinducers.

It is another objective of this disclosure to formulate compositions for use in disrupting a biofilm formed by a microbial population on a surface due to adhesions.

It is a still further objective of this disclosure to formulate compositions for use to improve antimicrobial activity within biofilm structures, including in embodiments where combinations of poloxamer surfactants provide micelles to deliver preservative systems to time release into the biofilm.

It is a still further objective of this disclosure to formulate compositions for use to improve antimicrobial activity within biofilm structures, including in embodiments where compositions utilize cationic minerals in medicinal clays for enhanced biofilm disruption and removal.

Other objects, embodiments and advantages of this disclosure will be apparent to one skilled in the art in view of the following disclosure, the drawings, and the appended claims.

### SUMMARY

The present disclosure is directed to antimicrobial compositions and methods of dissolving biofilm caused by the release of autoinducers, virulence factors and complicated by adhesion molecules and pathogen resistance. According to certain embodiments, an antimicrobial composition of the present disclosure comprises at least one nonionic block EO-PO copolymer, at least one preservative system and/or medicinal clay, and polyacrylamide. In some embodiments, the composition comprises at least two or at least three nonionic block EO-PO copolymers. In some embodiments, the nonionic block EO-PO copolymers are poloxamers, including poloxamer 188, poloxamer 338, and/or poloxamer 407. In some embodiments, the preservative system comprises phenoxyethanol and/or octenidine. Beneficially, in some embodiments the three poloxamers and polyacrylamide work synergistically to block the attachment of bacteria and further combine to kill embedded pathogens via the delivery of octenidine by the non-ionic activity of at least one of the polymers, while reestablishing a three-dimensional matrix for cell migration.

In certain embodiments, the combined molecular weight of the nonionic EO-PO copolymers is at least 11,000 g/mol, at least 13,000 g/mol, or at least 14,000 g/mol. In certain embodiments, the nonionic block EO-PO copolymers form micelles and the preservative system is packaged within the micelles. Beneficially, the viscosity of the composition may increase once applied to a wound. In some embodiments, the viscosity of the composition is at least about 100 times higher at about 37° C. than the viscosity of the composition at about 20° C.

Methods of disrupting a biofilm matrix are also provided. In some embodiments, the method comprises contacting the biofilm matrix with a composition comprising at least one, at least two, or at least three nonionic block EO-PO copolymers, at least one preservative system, and polyacrylamide. In some embodiments, the nonionic block EO-PO copolymers are poloxamers, including poloxamer 188, poloxamer 338, and/or poloxamer 407. The nonionic block EO-PO copolymers form micelles and/or mixed micelles that function to dissolve the biofilm matrix and deliver the preservative to the microbial population. Beneficially, once delivered, the preservative system kills and/or inhibits the microbial population. In some embodiments, two preservative systems are used. In some embodiments, the preservative system comprises phenoxyethanol and/or octenidine.

Methods of treating a subject with a composition are also provided. In some embodiments, the method comprises administering to a tissue or organ of a subject in need of treatment an antimicrobial composition according to the present disclosure and reducing microbial populations on the subject and/or removing biofilm from the tissue or organ in need of treatment. In some embodiments, the tissue is a wound, including a chronic wound. In certain embodiments, the composition is applied topically to the tissue. In certain embodiments, the viscosity of the composition is at least about 100 times higher following administration to the tissue or organ as compared to the viscosity of the composition prior to administration.

Methods of inhibiting quorum sensing in a population of bacteria are also provided. In some embodiments, the method comprises contacting the population of bacteria with a composition of the present disclosure, wherein the composition inhibits and/or decreases the release of autoinducers from the bacteria and/or attach to autoinducer receptors without activating the receptors, thereby inhibiting bacterial quorum sensing.

Methods of interfering with biofilm cation signaling are also provided. In some embodiments, the method comprises contacting a population of bacteria in a biofilm with a composition of the present disclosure, wherein the composition interferes with cation signaling to reduce or eliminate biofilm.

Methods of treating epidermolysis bullosa in a subject are also provided. In certain embodiments, the method comprises topically administering to a tissue of the subject a composition of the present disclosure. In certain embodiment, the tissue is skin. In certain embodiments, the administering step comprises spraying.

While multiple embodiments are disclosed, still other embodiments will become apparent to those skilled in the art from the following detailed description, which shows and describes illustrative embodiments. Accordingly, the drawings and detailed description are to be regarded as illustrative in nature and not restrictive.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts the logarithmic increase in dynamic viscosity of an antimicrobial composition as temperature increases.

FIG. 2 shows the progression of healing of a chronic wound treated with an antimicrobial composition of the present disclosure, as described in Example 5, Case Study 1.

FIG. 3 shows the progression of healing of a chronic wound treated with an antimicrobial composition of the present disclosure and a commercially available silver-based composition, as described in Example 5, Case Study 2.

FIG. 4 shows the progression of healing of a chronic wound treated with an antimicrobial composition of the present disclosure and a commercially available silver-based composition with bacterial autofluorescence imaging on day 0 (pre-treatment), day 12, and day 34, as described in Example 5, Case Study 3.

FIG. 5 shows the progression of healing of a chronic wound treated with an antimicrobial composition of the present disclosure and a commercially available silver-based composition with bacterial autofluorescence imaging on day 0 (pre-treatment) and 24 hours, as described in Example 5, Case Study 4.

FIG. 6 shows the progression of healing of a chronic wound treated with an antimicrobial composition of the present disclosure and a commercially available silver-based composition with bacterial autofluorescence imaging on day 0 (pre-treatment) and 36 days, as described in Example 5, Case Study 6.

FIG. 7 shows the progression of healing of a chronic wound treated with an antimicrobial composition of the present disclosure and a commercially available silver-based composition with bacterial autofluorescence imaging on day 0 (pre-treatment), day 12, and day 26, as described in Example 5, Case Study 7.

FIG. 8 is a chart showing the AI-2 assay results as described in Example 9. The test product is a diluted sample (25%) of a composition of the present disclosure and (75%) agrobacterium minimal medium (AB).

Various embodiments of the present disclosure will be described in detail with reference to the drawings, wherein like reference numerals represent like parts throughout the several views. Reference to various embodiments does not limit the scope of the disclosure. Figures represented herein are not limitations to the various embodiments according to the disclosure and are presented for exemplary illustration of the invention.

### DETAILED DESCRIPTION

The present disclosure is not to be limited to that described herein. Mechanical, electrical,

It is further to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form. Numeric ranges recited within the specification are inclusive of the numbers within the defined range. Throughout this disclosure, various aspects are presented in a range format. It should be understood that the description in range format is merely for convenience and brevity and should not be construed as an inflexible limitation on the scope of the disclosure and the invention. Accordingly, the description of a range should be considered to have specifically disclosed all the possible sub-ranges as well as individual numerical values within that range (e.g., 1 to 5 includes 1, 1.5, 2, 2.75, 3, 3.80, 4, and 5).

As used herein, the term “and/or”, e.g., “X and/or Y” shall be understood to mean either “X and Y” or “X or Y” and shall be taken to provide explicit support for both meanings or for either meaning, e.g., A and/or B includes the options i) A, ii) B or iii) A and B.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

So that the present disclosure may be more readily understood, certain terms are first defined. Unless defined otherwise, all technical and scientific terms used herein have the same meaning as commonly understood by one of ordinary skill in the art to which embodiments of the disclosure pertain. The definitions are provided to aid in describing particular embodiments, and are not intended to limit the claimed invention, because the scope of the invention is limited only by the claims. Many methods and materials similar, modified, or equivalent to those described herein can be used in the practice of the embodiments without undue experimentation, but the preferred materials and methods are described herein. In describing and claiming the embodiments, the following terminology will be used in accordance with the definitions set out below.

The term “about,” as used herein, refers to variation in the numerical quantity that can occur, for example, through typical measuring and liquid handling procedures used for making concentrates or use solutions in the real world; through inadvertent error in these procedures; through differences in the manufacture, source, or purity of the ingredients used to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

The term “actives” or “percent actives” or “percent by weight actives” or “actives concentration” are used interchangeably herein and refers to the concentration of those ingredients involved in cleaning expressed as a percentage minus inert ingredients such as water or salts.

The term “antimicrobial” is defined herein to mean a composition that inhibits the growth of, or kills, microbes including bacteria, protozoans, viruses, yeast, fungi, or other infectious agents.

The term “anti-pathogenic” is defined herein to mean a composition that inhibits the growth of, or kills, organisms including pathogens, protozoans, viruses, yeast, fungi, or other infectious agents.

The term “biofilm,” as used herein, means an extracellular matrix in which a population of microorganisms, microbes, or pathogens are dispersed and/or form colonies. Biofilms are understood to be typically made of polysaccharides and other macromolecules, often referred to as exopolysaccharides, that are concentrated at an interface (usually solid/liquid) and act as a binding agent that surrounds such populations of microorganisms. Biofilms are further understood to include complex associations of cells, extracellular products and detritus (or non-living particulate organic material) that are trapped within the biofilm or released from cells within the biofilm. The term biofilm, as used herein, further refers to the ASTM definition of biofilm as an accumulation of bacterial cells immobilized on a substratum and embedded in an organic polymer matrix of microbial origin. Biofilms are understood to be a dynamic, self-organized accumulation of microorganisms and microbial and environmental by-products that is determined by the environment in which it lives.

As used herein, the term “microbe” refers to any noncellular or unicellular (including colonial) organism. Microorganisms include all prokaryotes. Microbes include bacteria (including cyanobacteria), spores, lichens, fungi, protozoa, virinos, viroids, viruses, phages, and some algae. As used herein, the term “microorganism” and is synonymous with microorganism.

The terms “include” and “including” when used in reference to a list of materials refer to but are not limited to the materials so listed.

As used herein, the term “exemplary” refers to an example, an instance, or an illustration, and does not indicate a most preferred embodiment unless otherwise stated.

As used herein, the term “treat”, “treated”, “treatment”, “treating” or like terms when used with respect to a disease or disorder refers to a therapeutic or prophylactic treatment that increases the resistance of a subject to development of the disease (e.g., to infection with a pathogen, such as a bacteria or fungus), that decreases the likelihood that the subject will develop the disease (e.g., become infected with the pathogen), that increases the ability of a subject that has developed disease (e.g., a pathogenic [e.g., fungal] infection) to fight the disease (e.g., reduce or eliminate at least one symptom typically associated with the infection) or prevent the disease from becoming worse, or that decreases, reduces, or inhibits at least one function of the pathogen, and/or to grow by at least 10% (e.g., at least 20%, 30%, 40%, 50%, 60%, 70%, 80%, 90%, 95%, or 100%). In some embodiments, “treat,” “treated,” “treatment” or “treating” refers to a therapeutic or prophylactic treatment that reduces microbial populations on a tissue that is infected.

The term “weight percent,” “wt. %,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt. %,” etc.

The term “subject” as used herein refers to any living being that would benefit from the compositions and methods described herein. For example, the subject may be an animal, including a human, avian, bovine, canine, equine, feline, hircine, lupine, murine, ovine, and porcine animal. Subjects may also be domesticated animals such as cats, dogs, rabbits, guinea pigs, ferrets, hamsters, mice, gerbils, horses, cows, goats, sheep, donkeys, pigs, and the like. In certain embodiments, the subject is a human.

The methods and compositions may comprise, consist essentially of, or consist of the components and ingredients as well as other ingredients described herein. As used herein, “consisting essentially of” means that the methods and compositions may include additional steps, components or ingredients, but only if the additional steps, components or ingredients do not materially alter the basic and novel characteristics of the claimed methods and compositions.

### Antimicrobial Compositions

According to embodiments, the nonionic block EO-PO copolymer(s) actively diffuse the preservative system into a biofilm matrix by dissolving the extracellular polymetric substances allowing for the transport of the preservative system into the biofilm bacteria membrane. In some embodiments, the preservative system enters the biofilm via diffusion. While not being limited by a method of action, the components of the composition work together to dissolve extracellular polymeric substances of the biofilm and pathogen membranes caused by the release of autoinducers, virulence factors and complicated by adhesion molecules and pathogen resistance. This dissolution allows for the delivery of the preservative system into the pathogen cytosol, causing cellular death.

Furthermore, the composition can interrupt, decrease, and/or inhibit bacterial quorum sensing. While not being limited by a method of action, the composition can act by disrupting bacterial anchoring which is necessary for quorum sensing and the formation of biofilms.

According to certain embodiments, an antimicrobial composition of the present disclosure comprises at least one nonionic block EO-PO copolymer, at least one preservative system, and polyacrylamide. In some embodiments, the composition comprises at least two or at least three nonionic block EO-PO copolymers. In some embodiments, the nonionic block EO-PO copolymer is a poloxamer, including poloxamer 188, poloxamer 338, and/or poloxamer 407. In some embodiments, the preservative system comprises phenoxyethanol and/or octenidine. The antimicrobial compositions can further include a carrier, such as water, and/or additional functional ingredients and/or delivery systems. Exemplary antimicrobial compositions are shown in Table 1. While the components may have a percent active of 100%, it is noted that the Tables do not recite the percent actives of the components, but rather, recites the total weight percentage of the raw materials (i.e., active concentration plus inert ingredients).

TABLE 1
Antimicrobial Compositions

| | First | Second | Third |
|---|---|---|---|
| | Exemplary | Exemplary | Exemplary |
| Material | Range wt.-% | Range wt.-% | Range wt.-% |
| Nonionic Block EO-PO | 1-50 | 5-30 | 10-25 |
| Copolymer | | | |
| Preservative System | 0.05-5 | 0.075-3 | 0.1-2 |
| Polyacrylamide | 0.01-5 | 0.1-3 | 0.25-2 |
| Carrier (e.g., water) | 0-98 | 20-80 | 50-80 |
| Additional Functional | 0-70 | 0-50 | 0-25 |
| Ingredients | | | |

In some embodiments, the pH of the composition is from about 2.0 to about 6.5, from about 3.5 to about 5.5, from about 3.75 to about 5.25, from about 4.0 to about 5.0, from about 4.0 to about 4.5, or any intervening range therein. In some embodiments, the pH is from about 4.0 to about 5.0. In some embodiments, the pH is less than about 4.5.

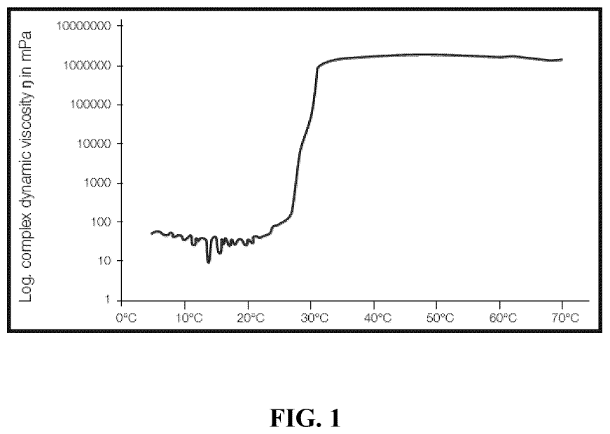

In certain embodiments, the composition may have non-Newtonian and/or thermos-reversible characteristics. The composition may be fluid when at room temperature (about 20° C.) but become a thick, stretchable gel or hydrogel when warmed to body temperature (about 37° C.). As shown in FIG. 1, in certain embodiments the composition has a lower viscosity when at temperatures from about 0° C. to about 30° C., but the viscosity increases dramatically at temperatures above about 30° C. The viscosity of the composition may be at least about 3 times higher, at least about 10 times higher, at least about 100 times higher, or at least about 1000 times higher at about 37° C. than the viscosity of the composition at about 20° C. Beneficially, the composition's low viscosity at room temperature allows for easy storage and application of the compositions. Once the composition is applied to a wound or warm surface, it quickly becomes a thicker, stretchable, and moldable hydrogel that form-fits the wound or surface. This beneficially allows the composition to fill tunnels and pores that would otherwise have to be packed or else missed. These characteristics save time, money and ensures a perfect form-fitting wound bed moisture system.

In certain embodiments, the composition is used at a temperature range of from about 20° C. to about 40° C., from about 25° C. to about 35° C., from about 30° C. to about 35° C., or any range therein. In some embodiments, use and/or storage at a temperature range of from about 25° C. to about 40° C. is ideal for composition stability. In some embodiments, the composition is cold processed at about 25° C. and used in wound environments from about 35° C. to about 40° C.

The antimicrobial composition can be provided as a liquid, hydrogel, gel, alginate gel, gel sheet, foam, emulsion, suspension, paste, cream, collagen, gauze, artificial tissue, hydrocolloid, ointment, or the like. In certain embodiments, the composition is provided as a hydrogel that is liquid at room temperature (about 20° C.). In some embodiments, the composition is provided as a ready to use composition, meaning that the composition is provided in a way that can be applied without needing to dilute it first.

The compositions may be packaged in any suitable container, such as flasks or bottles, including squeeze-type or pump bottles, as well as bottles provided with a spray apparatus (e.g. trigger spray or push-type spray) which is used to dispense the composition by spraying. In some embodiments, the composition is provided in a sachet that can be torn or cut open.

The composition may be provided in various packaging sizes. Non-limiting examples of packaging sizes include 10 g, 30 g, or 50 g sachets or 1 oz, 1.5 oz, 1.7 oz, 3 oz, 500 ml and 1-liter bottles.

Beneficially, compositions of the present disclosure are gentle to the skin and non-damaging to tissues. Compositions of the present disclosure can gently dissolve biofilms without the use of harsh chemicals or antibiotics. The compositions not only dissolve existing biofilms but are also capable of inhibiting and/or preventing the continued formation of biofilms.

The composition of the present disclosure reverses inflammation that impairs microvascular perfusion necessary to reverse ischemia and nutrient depravation in chronic wounds. The blend improves microvascular perfusion by increasing blood flow and reducing vascular resistance. This improvement in microvascular perfusion is due to reduce the adhesion of white blood cells to endothelial cells, which can obstruct blood flow. By reducing the expression of adhesion molecules on both leukocytes and endothelial cells the blend revitalizes ischemic tissue and provides for an enhanced immune system capable of participating fully in the reduction of bioburden and the inanition of wound closure.

Traditional wound treatments such as antibiotics and antiseptics, even with more potent dosing, are not effective against pathogens that are encapsulated in biofilms. This is because they are designed to attack the pathogen when it is at its strongest. While not limited to a method of action, compositions of the present disclosure are effective against infections because they act against pathogens within biofilms that are either less metabolically active or quiescent. The pathogen doubling rate is greatly reduced in such quiescent pathogens, however the virulence of the pathogens becomes substantially greater. The present disclosure is directed toward reducing virulence factors and dissolving biofilms as a method to reduce infection.

As a result of changes in gene expression, the microorganisms within biofilms tend to enter a dormant or slow-growing state, which makes them more resistant to antibiotics and other antimicrobial agents due to reduced doubling rates. When bacteria become metabolically inactive within biofilms, they become more virulent and capable of spreading quickly. This is because the extracellular polymetric matrix that surrounds the bacteria acts as a protective barrier, preventing the immune system and antimicrobial agents from reaching the microorganisms. As a result, the bacteria within the biofilm can continue to grow and produce virulence factors that contribute to the persistence of the infection.

One of the primary mechanisms by which bacteria within biofilms deploy virulence factors is through the production of extracellular vesicles (EVs). EVs are small membrane-bound vesicles that are released by the bacteria and contain a variety of cargo, including virulence factors. These vesicles can be released from the biofilm and transported to other areas of the host or environment, where they interact with and damage host cells. EVs produced by bacteria within biofilms have been shown to contain a variety of virulence factors, including toxins such as pyocyanin, rhamnolipids, and lipopolysaccharides. These toxins cause damage to host cells by disrupting membrane integrity, generating reactive oxygen species, and inducing cell death. Additionally, some biofilm-forming bacteria produce biofilm-specific toxins, such as the extracellular matrix-degrading enzyme dispersin B, which can cause damage to the extracellular matrix of host tissues.

As an example, P. aeruginosa is one of the most common bacterial species found in chronic wound biofilms. It uses EVs to transport the virulence factor elastase. It is a significant contributor to the pathogenesis of chronic wound infections. Elastase is a protease that can degrade a variety of extracellular matrix proteins, including elastin, collagen, and fibronectin. The release of elastase from within chronic wound biofilms can cause damage to host tissues, disrupt the healing response, and promote the persistence of infection.

Compositions of the present disclosure target virulence factors as a method of reducing infections and damage to host cells and their extracellular matrix support proteins.

### Nonionic Block EO-PO Copolymers

Compositions of the present disclosure comprise one or more nonionic block EO-PO copolymer(s) (also includes poly (ethylene oxide) (PEO)-poly (propylene oxide) (PPO) copolymers). The polymers provide beneficial antimicrobial properties as they can effectively carry pharmaceutical compounds, such as additional therapeutic agents, into the pathogens, thereby aiding in killing the pathogens.

Nonionic block EO-PO copolymers of the present disclosure are surfactant copolymers comprised of both polyethylene oxide (PEO) and polypropylene oxide (PPO) blocks, with the balance of specific hydrophilic and lipophilic components dictating their amphiphilicity and surface activity. Naturally present as unimers, the copolymers self-assemble into micelles when mixed in solutions above their critical micelle concentration, also referred to herein as “critical mass concentration”. Consisting of a hydrophobic core and hydrophilic outer shell, considerable amounts (e.g., 20-30% wt./vol) of water insoluble compounds can be packaged within these micelles. Thus, nonionic, non-cytotoxic block EO-PO copolymers can make effective delivery vehicles for other compounds, such as a preservative system of the present disclosure.

In embodiments, the nonionic block EO-PO copolymers include, but are not limited to, poloxamers. These poloxamers are odorless, tasteless, white, waxy granules with free-flowing properties. Poloxamers are amphiphilic in nature, as they are soluble in both polar and nonpolar solvents. The amphiphilic properties stem from a tri-block configuration, consisting of a hydrophobic unit [poly (propylene oxide) (PPO)] in between two hydrophilic units [poly (ethylene oxide) (PEO)] with the basic sequence of A-B-A and having the structure (PEOₐ-PPO_{b}-PEOₐ) shown below:

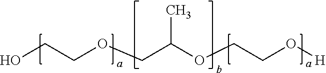

Exemplary poloxamers and the number and average size of EO/PEO and PO/PPO blocks include those shown by Chowdhury P. et al., Pluronic Nanotechnology for Overcoming Drug Resistance. In: Yan B. et al. (eds) Bioactivity of Engineered Nanoparticles (2017), which is reproduced below as Table 2:

TABLE 2

| | Average | | |
|---|---|---|---|
| | Molecular | No. | No. |
| | Weight | of EO | of PO |
| Name | (g/mol) | units | units |
| Poloxamer 124 (Pluronic ® L-44) | 2090-2360 | 12 | 20 |
| Poloxamer 188 (Pluronic ® F-68) | 7680-9510 | 80 | 27 |
| Poloxamer 237 (Pluronic ® F-87) | 6840-8830 | 64 | 37 |
| Poloxamer 338 (Pluronic ® F-108) | 12700-17400 | 141 | 44 |
| Poloxamer 407 (Pluronic ® F-127) | 9840-14600 | 101 | 56 |
| Poloxamer 401 (Pluronic ® L121) | 4400 | 10 | 68 |
| Poloxamer 184 (Pluronic ® L-64) | 2900 | 26 | 30 |

Additional exemplary poloxamers include Poloxamer 105 having a molecular structure: polymer with oxirane (11;16) where oxirane means ethylene oxide, also described as EO₂₇PO₅₆EO₂₇. Further Poloxamer 85 has a molecular structure: triblock copolymer with central chain of poly(propylene oxide) (70 units) flanked by two hydrophilic chains of poly(ethylene oxide) (20 units), function: surfactant.

These EO-PO copolymers have the same chemical structure but differ in the number of EO/PEO and PO/PPO units, as well as in molecular weight. Due to the hydrophobic and hydrophilic nature of the poloxamers, the amphiphilic properties of poloxamers differ depending on the number of EO/PEO or PO/PPO units and can be determined by the hydrophilic-lipophilic balance (HLB) of the poloxamer.

In embodiments, the nonionic block EO-PO copolymer has the formula PEOₐ-PPO_{b}-PEOₐ, where the sum of a=70-160 and b=40-65 and/or has the formula PEOₐ-PPO_{b}-PEOₐ, where the sum of a=80-150 and b=20-60.

In other embodiments, suitable polymers include a di-block polymer comprising a PEO block and a PPO block, a center block of polyoxypropylene units (PPO), and having blocks of polyoxyethylene grafted onto the polyoxypropylene unit or a center block of PEO with attached PPO blocks. Further, this polymer can have further blocks of either polyoxyethylene or polyoxypropylene in the molecules. A suitable average molecular weight range of useful surfactant polymers can be about 1,000 to about 40,000 (molecular mass) and the weight percent content of ethylene oxide can be about 20-90 wt-%. In embodiments, the nonionic EO-PO copolymer having an average molecular weight of from about 5,000 to about 20,000 and a weight percent content of ethylene oxide of from about 50-80 wt % or from about 60-90 wt-%.

In further embodiments, the nonionic block EO-PO copolymers comprise, consist of or consist essentially of a nonionic block EO-PO copolymer having the formula PEOₐ-PPO_{b}-PEOₐ, where the sum of a=100-160 and b=30-50, such as poloxamer 338, a nonionic block EO-PO copolymer having the formula PEOₐ-PPO_{b}-PEOₐ, where the sum of a=80-120 and b=30-60, such as poloxamer 407, and/or a nonionic block EO-PO copolymer having the formula PEOₐ-PPO_{b}-PEOₐ, where the sum of a=70-100 and b=20-40, such as poloxamer 188. In some embodiments, the composition comprises poloxamer 338, poloxamer 407, and/or poloxamer 188. In certain embodiments, compositions of the present disclosure include all three of a nonionic block EO-PO copolymer having the formula PEOₐ-PPO_{b}-PEOₐ, where the sum of a=100-160 and b=30-50, a nonionic block EO-PO copolymer having the formula PEOₐ-PPO_{b}-PEOₐ, where the sum of a=80-120 and b=30-60, and a nonionic block EO-PO copolymer having the formula PEOₐ-PPO_{b}-PEOₐ, where the sum of a=70-100 and b=20-40. In some embodiments, the composition comprises poloxamer 338, poloxamer 407, and poloxamer 188.

In some embodiments of the disclosure, at least one of the included copolymers reaches critical mass concentration. In some embodiments, the copolymer micelles comprise a single copolymer. In other embodiments, the copolymer micelles may be mixed copolymer micelles comprising more than one copolymer. For example, the copolymer micelles may be comprised entirely of poloxamer 338, entirely of poloxamer 407, or entirely of poloxamer 188. Alternatively, and/or additionally, the copolymer micelles may comprise a combination of, for example, poloxamer 338 and poloxamer 407, poloxamer 338 and poloxamer 188, poloxamer 407 and poloxamer 188, or poloxamer 338, poloxamer 407, and poloxamer 188. Beneficially, a blend of poloxamers can increase the stability and size of the micelles formed, as well as improve encapsulation of the preservative system and release into the bacteria/pathogen cytosol.

The copolymers of the present disclosure have unique functions that make them particularly suitable for inclusion in an antimicrobial composition. A nonionic block EO-PO copolymer having the formula PEOₐ-PPO_{b}-PEOₐ, where the sum of a=100-160 and b=30-50, such as poloxamer 338, can combine with polyacrylamide to keep wound beds free of primary and secondary pathogen attachments and biofilms that can inhibit cell migration. While not being limited to a mechanism of action, a copolymer such as poloxamer 338 can coat the polyacrylamide 3D structure required for cell migration thereby inhibiting bacterial adhesions that would interfere with new cell migration and wound closure. Copolymers such as poloxamer 338 can furthermore be a polymer for critical mass concentration and can block pathogen adhesion to the wound bed or selected material. Additionally, these copolymers can deliver the octenidine (and/or other components of the preservative system) to the pathogen, which further destabilizes pathogen membranes.

A nonionic block EO-PO copolymer having the formula PEOₐ-PPO_{b}-PEOₐ, where the sum of a=80-120 and b=30-60, such as poloxamer 407, is particularly effective at taking therapeutic agents—including the octenidine preservative—into pathogens and killing the pathogens (i.e., “Trojan Horse” effect). While not limited to a method of action, copolymers such as poloxamer 407 and octenidine “pits” the membrane of the bacteria/pathogen and weakens the membrane enough for the octenidine to diffuse into the target and kill it without harm to the surrounding tissue. In some embodiments, this mode of action works synergistically with the other polymers.

A nonionic block EO-PO copolymer having the formula PEOₐ-PPO_{b}-PEOₐ, where the sum of a=70-100 and b=20-40, such as poloxamer 188, can also be used to form micelles that deliver the octenidine preservative. Copolymers such as poloxamer 188 are also particularly effective at cellular healing and bandaging broken host cell membranes and can aid in the repair of endothelial cell. These copolymers can plug the holes in the endothelial membranes with its hydrophobic tails and assists in the reorganization of endothelial membranes and the extracellular matrix which have become disorganized in the chronic wound environment. Further, copolymers such as poloxamer 188 reduce the expression of adhesion molecules on both leukocytes and endothelial cells. The blend revitalizes ischemic tissue and provides for an enhanced immune system capable of participating fully in the reduction of bioburden and the inanition of wound closure. The repair of endothelial cells allows for the improvement of vessels necessary for nutrient transport into the wound bed and the re-oxygenation of hypoxic cells accounting for increased survival rates necessary for wound closure. Beneficially, repairing wound beds can also reverse hypoxia, which is essential for wound repair. The re-organization of the extracellular matrix and the endothelial membranes is a critical aspect of cell migration and tissue regeneration. Additionally, copolymers such as poloxamer 188 can defend the extracellular matrix from escaping pathogens released from the extracellular polymeric substance. These copolymers work synergistically with polyacrylamide and copolymers such as poloxamer 338 to reestablish cell migration and initiate wound closure.

According to particular embodiments, inclusion of copolymers such as poloxamer 338, poloxamer 407, and poloxamer 188 in a composition provide unexpected benefits in biofilm disruption, cellular entry, and membrane healing.

In certain embodiments, at least two of the copolymers, or at least one of the copolymers and the polyacrylamide, work synergistically to build a 3D scaffolding system capable of transporting epithelizing host cells across the wound bed once the biofilm and bioburden have been removed. Beneficially, this 3D scaffolding system can provide an additional method for accelerated wound healing. While not limited to a method of action, it is believed that chronic wounds chronic wounds become two dimensional, which does not support cell migration. By forming a 3D polymer scaffolding system, stalled healing can be overcome. Beneficially, copolymer blends of the present disclosure support collagen and fibronectin allowing them to reorganize the extracellular matrix. Unexpectedly, the realignment of these support fibers combined with the 3D scaffolding system show advanced healing beyond expectations. In some embodiments, poloxamer 338 and polyacrylamide work synergistically to build the 3D scaffolding system.

In certain embodiments, the molecular weight of the nonionic EO-PO copolymer, or the combined molecular weight of the nonionic EO-PO copolymers, is at least 11,000 g/mol, at least 13,000 g/mol, or at least 14,000 g/mol. In certain embodiments, the combined molecular weight of the nonionic EO-PO copolymers is at least 14,000 g/mol. Beneficially, nonionic EO-PO copolymers with a combined molecular weight over 12,000 g/mol are more effective at removing biofilms as compared to nonionic EO-PO copolymers with a combined molecular weight less than 12,000 g/mol. While not limited to a method of action, in terms of molecular weight, a higher molecular weight poloxamer may have a stronger disruptive effect on biofilm than a lower molecular weight poloxamer. This is due to the fact a higher molecular weight poloxamer has more hydrophobic and hydrophilic blocks, which can better interact with the biofilm matrix and disrupt its structure.

Hydrophilic-Lipophilic Balance (HLB) is a measure of the relative hydrophilic and lipophilic properties of a surfactant molecule. The HLB value is a number between 0 and 20 for lipophilic surfactants, 20 to 40 for hydrophilic surfactants, and 10 to 18 for surfactants that are balanced between hydrophilicity and lipophilicity. The HLB value of a surfactant can be used to predict its behavior in a given system. Surfactants with high HLB values are more hydrophilic and tend to form water-in-oil emulsions, while those with low HLB values are more lipophilic and tend to form oil-in-water emulsions. The appropriate HLB value for a particular application depends on the nature of the system and the desired properties of the emulsion. For example, poloxamer 188 has an HLB of 24, poloxamer 338 has an HLB of 22.5, and poloxamer 407 has an HLB of 22. Poloxamers can interfere with autoinducer signaling in several ways. One mechanism involves the physical sequestration of autoinducers within the hydrophobic core of the micelles formed by poloxamers. This prevents the autoinducers from reaching their target receptors on bacterial cells, thereby inhibiting quorum sensing.

In certain embodiments, compositions of the present disclosure are also capable of dissolving membrane lipids, which improves biofilm removal. Due to their high molecular weight, copolymers of the present disclosure can interact with and disrupt the lipid bilayer of bacterial membranes, causing leakage of intracellular components and ultimately leading to cell death.

### Preservative System

Compositions of the present disclosure comprise at least one preservative system. In some embodiments, the composition comprises two preservative systems. In certain embodiments, the preservative system comprises phenoxyethanol and/or octenidine. The preservative systems, including for example octenidine, can include cationic and other antiseptics that are effective against a wide range of microorganisms, including bacteria and fungi.

In some embodiments the preservative system comprises a phenolic preservative, such as phenoxyethanol. Some other, non-limiting examples of phenolic preservatives include pentachlorophenol, orthophenylphenol, chloroxylenol, p-chloro-m-cresol, p-chlorophenol, chlorothymol, m-cresol, o-cresol, p-cresol, isopropyl cresols, mixed cresols, phenoxyethylparaben, phenoxyisopropanol, phenyl paraben, resorcinol, and derivatives thereof. Some non-limiting examples of halogen compounds include sodium trichloroisocyanurate, sodium dichloroisocyanurate, iodine-poly(vinylpyrolidinonen) complexes, and bromine compounds such as 2-bromo-2-nitropropane-1,3-diol, and derivatives thereof.

In some embodiments, the preservative system comprises phenoxyethanol. Phenoxyethanol is a hydrophilic preservative that can aid in the preservation of the composition and protection of the integrity of the composition within a wound. Phenoxyethanol provides an excellent defense for the composition in the extracellular matrix and supports the poloxamers, for example poloxamer 188, in repairing subject tissue cells.

Beneficially, phenoxyethanol can also exhibit antimicrobial properties and is effective against a wide range of pathogens, including gram-negative and gram-positive bacteria as well as Candida albicans. In some embodiments, phenoxyethanol works together with the other components of the composition to kill and/or inhibit pathogens.

In some embodiments, the preservative system comprises octenidine. Octenidine dihydrochloride is a preservative derived from pyridine that is active against gram-negative and gram-positive bacteria as well as Candida albicans. Beneficially, octenidine is not absorbed through the skin or mucous membranes, making it ideal for wound applications. While not being limited to a mechanism of action, octenidine destabilizes the pathogen membrane and enters the pathogen once the porosity of the membranes has been irreversibly damaged by the octenidine-polymer blend. Octenidine is thereby given “free passage” into the cytosol where it kills any pathogen able to survive the loss of membrane integrity.

In some embodiments, the composition comprises only phenoxyethanol or only octenidine. In other embodiments, the composition comprises both phenoxyethanol and octenidine.

In certain embodiments, the preservative system is “packaged” or encapsulated within the nonionic block EO-PO copolymer micelles. In embodiments comprising two or more preservative systems, all preservatives may be encapsulated in the micelles or one or more may be encapsulated and the others delivered freely (i.e., not encapsulated within a micelle). For example, in embodiments comprising both phenoxyethanol and octenidine, both phenoxyethanol and octenidine can be encapsulated within the micelles or only octenidine or only phenoxyethanol can be encapsulated within the micelle and the other preservative may be delivered freely. In certain embodiments, octenidine is encapsulated within the copolymer micelles and phenoxyethanol is delivered freely so as to preserve the composition and protect the composition integrity. In certain embodiments, the octenidine is delivered in micelles comprising a combination of the nonionic block EO-PO copolymer, e.g. poloxamer 188, poloxamer 407, and/or poloxamer 338. Beneficially, the nonionic block EO-PO copolymer (e.g. poloxamer) micelles time release the preservative system (e.g. octenidine and/or phenoxyethanol) into the biofilm and pathogens within the biofilm.

The micelles improvement of the delivery of the preservative system (e.g. octenidine) through several mechanisms, including solubility, penetration, release, and antimicrobial activity enhancement to improve effectiveness in combating biofilm-related infections.

Without being limited to a particular mechanism of action, the micelles improve delivery of the preservative system according to various mechanisms of action as summarized herein (utilizing octenidine as an exemplary component of the preservative system):

Solubilization and stability: octenidine is a hydrophobic compound with limited solubility in water. Poloxamer micelles solubilize the hydrophobic octenidine by incorporating it into their hydrophobic cores. This solubilization increases the aqueous solubility of octenidine, enabling its dispersion in aqueous solutions and enhancing its stability.

Enhanced penetration into biofilms: Biofilms present a significant barrier to antimicrobial agents due to their complex structure and the presence of extracellular polymeric substances (EPS). Poloxamer micelles improve the penetration of octenidine into biofilms by encapsulating it within their hydrophobic cores. The poloxamer micelles act as carriers, facilitating the transport of octenidine through the EPS matrix and increasing its contact with the microorganisms within the biofilm. This provides enhanced membrane disruption of the lipid bilayer, compromising the integrity and function of the microbial cell membrane. As a result, the microorganisms become more susceptible to the octenidine that targets the compromised membrane.

Sustained release: Poloxamer micelles provide controlled and sustained release of octenidine. The encapsulated octenidine within the poloxamer micelles is released gradually over time, ensuring a prolonged exposure of the biofilm microorganisms to the octenidine. This sustained release profile enhances the effectiveness of octenidine by maintaining an optimal concentration of octenidine for an extended period, which is particularly beneficial for combating persistent biofilm infections.

Increased contact time: The presence of poloxamer micelles prolongs the contact time between octenidine and biofilm microorganisms. Poloxamer micelles adhere to the biofilm surface or become trapped within the extracellular polymeric substances (EPS) matrix. This sustained interaction allows the octenidine to exert its activity over an extended period, enhancing their effectiveness against the biofilm microorganisms.

Synergistic effects: Poloxamer micelles enhance the antimicrobial activity of octenidine through synergistic effects. Some of the poloxamer micelles possess intrinsic antimicrobial properties due to their surfactant nature. When combined with octenidine, the poloxamer micelles exhibit synergistic effects, resulting in a greater antimicrobial effect than the individual components alone. This synergistic interaction improves the overall efficacy of octenidine in eliminating biofilm-associated microorganisms.

Molecular weight: While not limited to a method of action, in terms of molecular weight, a higher molecular weight poloxamer may have a stronger disruptive effect on biofilm than a lower molecular weight poloxamer. This is due to the fact a higher molecular weight poloxamer has more hydrophobic and hydrophilic blocks, which can better interact with the biofilm matrix and disrupt its structure.

### Polyacrylamide

Antimicrobial compositions of the present disclosure further comprise polyacrylamide. While not being limited to a mechanism of action, polyacrylamide is helpful for tissue engineering and can build a 3D scaffolding system to allow for cell migration in wound beds that have been cleared of biofilm. In some embodiments, the polyacrylamide works together with one or more of the nonionic block EO-PO copolymers to keep the wound bed free of pathogen attachments and/or create the 3D scaffolding system.

In some embodiments, the polyacrylamide is cross-linked with N,N-methylenebisacrylamide which enhances the structural function of the compound. While not limited to a method of action, cross-linking to methylenebisacrylamide ensures polyacrylamide can form the 3D scaffolding system.

### Medicinal Clays

Medicinal clays include at least one source of clay to provide cation sources for the treatment of biofilm and wounds as described herein. Suitable clays include, but are not limited to, a natural clay or clay mineral and/or synthetic clay or clay mineral, or other suitable materials having clay-like properties, so long as the clay is medicinal and provides effective anti-pathogenic efficacy. In embodiments the medicinal clay provides a cation source. In embodiments, a suitable clay includes a blend of minerals and nutrients that cleanse and aid in the antimicrobial effectiveness of silver and other medicants. A combination of clays can be employed as well.

Clays include a variety of natural mineral made up of crystalline material. Clay minerals have a sheet-like structure and are composed of mainly silicate and aluminate groups. In some embodiments, the medicinal clays are predominantly layered silicate structures. Exemplary silicate classifications include phyllosilicates (e.g., 2:1 phyllosilicates, 1:1 phyllosilicates (kaolinites and serpentines), etc.), smectite, illite, illite-smectite, kaolinite, and other silicates, e.g., aluminum silicates, magnesium aluminum silicates, magnesium trisilicates, and the like. Various embodiments of the medicinal clays have layered silicate structures as further described herein.

In preferred embodiments, the medicinal clay is referred to as a Fentonite™ medicinal clay, which include phyllosilicates (including for example bentonites, rectorites, and other clays) having one or more, two or more, or at least three, at least four, or all five of the following product specifications: Cation Exchange Capacity (CEC)>10 mEq/100 g, preferably from about 10 mEq/100 g to about 100 mEq/100 g; Oxidation-Reduction Potential (ORP)>250 mV>300 mV, or preferably >400 mV; pH<5.0 (or between about 2.5 and about 5); Total Combined Illite/Smectite/I-S>40%; a 2:1 tetrahedral/octahedral phyllosilicate with reduced iron octahedral and exchangeable cations; contains at least about 1% pyrite (iron), preferably between about 1-20% pyrite (iron); and/or Heavy Metals (total)<40 ppm.

In still further embodiments the clay has a Cation Exchange Capacity of at least about greater than about 10 mEq/100 g, preferably from about 10 mEq/100 g to about 100 mEq/100 g and an Oxidation-Reduction Potential greater than about 250 mV, >300 mV, or preferably >400 mV. Without being limited to a particular mechanism of action, the clay having the defined CEC and ORP provides a synergy in efficacy of the medicinal clay composition.

In exemplary embodiments, the medicinal clay can be a naturally mined medicinal (namely anti-pathogenic) clay referred to as Fentonite™, wherein the clay is a 2:1 tetrahedral/octahedral phyllosilicate with reduced iron octahedral and exchangeable cations and is therefore capable of catalyzing Fenton reactions, namely catalyzing Fenton reactions within fluids, such as bacterial cytosol and macrophage lysosomes where the medicinal clay will be delivered for the treatment methods described herein. As referred to herein the 2:1 phyllosilicate describes the crystalline structure of the clay itself (2:1 tetrahedral-octahedral-tetrahedral clay) and can include a variety of illites, smectites, and illite-smectite mixed clays (which are referred to as rectorites clays having alternative layers of the illite-smectite), along with other silicates, chlorites, and smectites (e.g., montmorillonites), and the like. The clay having reduced octahedral and exchangeable cations refers to the active component that conveys the medicinal, i.e., antimicrobial, effects, namely the presence of reduced metallic cations in both the interlayer spaces and within the octahedral crystalline layer of the clay (including but not limited to iron, aluminum, and magnesium). The crystalline structure of the clay beneficially holds, protects, and releases the reduced metallic cations under desired conditions, including those delivery conditions described herein.

In embodiments the medicinal clay has a CEC, or amount of cations that can be held in the clay material, as a raw material that needs to be maintained through processing and manufacturing as well as prior to application on the end user. Research indicates that a CEC of greater than 10 mEq/100 grams is needed for anti-pathogenic effect. Typically, the CEC of illite-smectite (I-S) clays varies from about 20-60 mEq/100 g, providing an exchange capacity of 10-20 mM/100 g for divalent cations.

The efficacy of clays is not only dependent on the CEC, but also on the oxidative state of those cations. Soluble reduced cations like Fe³⁺ and Fe²⁺ and Al³⁺ contribute to the non-cytotoxic and anti-pathogenic mechanism of clays by interacting with proteins, enzymes, nucleic acids, and negatively charged (anionic) compounds in the cytosol and membranes of pathogens. In an embodiment, the soluble reduced cations chelate anions. Once inside the pathogen's membranes and cytosol, they become oxidized and contribute to misfolding of proteins, enzyme deactivation, precipitate formation, membrane oxidation, and initiation of redox pathways that produce reactive oxygen species (ROS), namely hydroxyl radicals, that attack pathogenic intracellular proteins and DNA.

Cations must be in their reduced form to exhibit anti-pathogenic effects, so it is essential that metallic cations have not been oxidized before they get to their intended site of action. An ORP/Eh of greater than 250 mV is required for anti-pathogenic effects. In preferred embodiments an ORP from about 300-600 mV is used. Beneficially, the clay provides cations for a non-cytotoxic and anti-pathogenic activity.

An acidic pH (<4.7) is also essential for the release of reduced cations from clay material in high enough concentrations to exhibit anti-pathogenic activity. Clays with a pH<3 that meet the above criteria for CEC and ORP have exhibited the most potent anti-pathogenic activity, however consideration needs to be given to the application of the product to determine if the pH is safe and/or tolerable for that application.

The particle size of the medicinal clay may be an important factor that can affect its effectiveness, as well as bioavailability, blend uniformity, segregation, and flow properties. In general, smaller particle sizes of clay increase its effectiveness by increasing the surface area. In some embodiments, the particle size of the clay is reduced through processes such as milling. In an embodiment, milling can be used to reduce the particle size of clay down to less than 100 microns. In additional embodiments it is desirable to prill the clay, such as prilling into bead-like structures (i.e., prilled beads).

In various embodiments, the average particle size of the clay is less than about 250 microns in diameter, less than about 100 microns in diameter, or less than about 90 microns in diameter, or less than about 80 microns in diameter, or less than about 70 microns in diameter, or less than about 60 microns in diameter, or preferably less than about 50 microns in diameter. In some applications, the average particle size of the clay is between about 10 to about 100 microns, between about 10 to about 50 microns, or between about 10 to about 25 microns in diameter. Without being limited to a particular mechanism of processing the clay for the compositions described herein, the clay particles pass through a mesh screen to achieve a uniform desired micron size, as is referred to as clay milling. As opposed to conventional use of a metal ball to aid in the milling, the compositions described herein are produced using a ceramic ball to ensure no metal contaminants are included.

In embodiments the medicinal clays can be hydrated. In further embodiments, the medicinal clays are polycationic compounds. In still further embodiments, the medicinal clays are amorphous and do not have a rigid structure.

In exemplary embodiments, the medicinal clay is a mined medicinal clay. Despite significant reporting of the anti-pathogenic efficacy and medicinal nature of various clays, their physical makeup is highly inconsistent and difficult to reproduce. Applicants describe herein that medicinal clay is dominated by phyllosilicates, namely smectites, illites, and illite-smectite (a group of clay minerals having an expandable interlayer structure). It has been identified that the expandable smectite interlayer region functions like a reservoir from which metals, which may have antimicrobial effects, are slowly released via cation exchange.

In a further embodiment, the medicinal clay composition has at least about 50 wt-% Smectite, Illite, and/or Illite-Smectite. In still further embodiments, the medicinal clay composition has at least about 60 wt-% Smectite, Illite and/or Illite-Smectite. In still further preferred embodiments, any of the medicinal clay compositions described herein also has a cation exchange capacity (CEC) of at least about >10 mEq/100 g, or between about 10 mEq/100 g to about 100 mEq/100 g. The CEC refers to the ability of the clay to hold onto cations (e.g., positively charged ions such as calcium (Ca²⁺), magnesium (Mg²⁺), and potassium (K⁺), sodium (Na⁺), silver (Ag⁺), hydrogen (H⁺), aluminum (Al³⁺), iron (Fe³⁺ and Fe²⁺), manganese (Mn²⁺), zinc (Zn²⁺) and copper (Cu²⁺)).

In a further embodiment, the medicinal clay has a transition metal combination that includes a level of pyrite ranging from about 1 wt-% to about 10 wt-% or a level of pyrite ranging from about 1 wt-% to about 5 wt-%. Beneficially, the natural minerals can release soluble transition metals at low pH which are effective in killing pathogens due to the generation of reactive oxygen species and damage to pathogen membranes.

The medicinal clay may also be modified with various substituents to alter the properties of the clay. Non-limiting examples of modifications include modification with organic material, polymers, reducing agents, and various elements such as sodium, iron, silver, or bromide, or by treatment with a strong acid. In some embodiments, a medicinal clay of the present disclosure is modified with reducing metal oxides. In preferred alternatives of the embodiments, when a medicinal clay is modified with reducing metal oxides, the medicinal clay is modified with pyrite. In still other embodiments, the medicinal clay is unmodified.

In some embodiments the medicinal clay can also have one or more of the following product specifications: color/appearance that is light tan to blue/gray/green, Smectite concentration between about 10-40%, Illite concentration between about 10-40%, Illite-Smectite (I-S) concentration between about 20-50%, Aluminum (Al₂O₃) concentration between about 10-30%, elemental concentration between about 5-15%, Iron (pyrite) concentration between about 4-20%, Iron (III) Oxide concentration between about 0-10%, Sulfur concentration between about 0-20%, Calcite concentration less than about 0.5%, Carbonate concentration less than about 0.5%, and/or Kaolinite concentration less than about 3.5%. Additional disclosure of clays is set forth in Unearthing the Antimicrobial Activity of a Natural Clay Deposit by Keith Morrison, Arizona State University (December 2015); Catalogued Dissertation Presentation, and https://core.ac.uk/download/pdf/4270172.pdf, each of which are herein incorporated by reference in its entirety.

An exemplary source of clay that can be used to provide anti-microbial clays include glacial clays including Kisameet Bay glacial clay. These clays can be modified with various substituents to alter the properties of the clay to the specifications described herein.

In exemplary embodiments, the medicinal clay can also be a synthetic clay that mimics the structure of the clay that provides the anti-pathogenic efficacy against pathogens. Synthetic clays can be used to overcome limitations of natural clays being highly heterogenous. Synthetic clays can have a crystalline composition of smectite, illite, and/or illite-smectite that is at least about 40 wt-% of the clay.

In some embodiments, synthetic clays having anti-pathogenic efficacy and medicinal efficacy should also have the predominate make-up as illite-smectite and pyrite, or smectite and pyrite. In some embodiments, the smectite and pyrite is at least about 75 wt-%, or from about 75-100 wt-% of the clay.

In embodiments, the synthetic clays also have the properties linked to ROS generation. It has been identified by Morrison et al., Nature Portfolio (2022) 12:1218 that pyrites in particular have mineral semi-conductor properties can be linked to ROS generation in solution.

In some embodiments, the medicinal clay is preferably sterilized after formulation into the clay delivery system to kill any environmental microbes in the clay. Methods of sterilization are used that do not negatively impact the stability or anti-pathogenic activity of the coated clay or delivery system. Similarly, in embodiments wherein a reducing agent may be added to an anti-pathogenic clay, the particle size of a reducing agent may also be an important factor that can affect its effectiveness, and in general, smaller particle sizes increase its effectiveness. Preferably, the average particle size of the reducing agent that may be added to an anti-pathogenic clay is less than 100 micron in size.

In various embodiments, the medicinal clay can be coated with or delivered in combination with the nonionic block EO-PO copolymer(s) (or other coatings e.g. mineral salts, polysaccharide polymers, etc. as disclosed in U.S. Publication No. 2023/0131873, which is incorporated herein by reference in its entirety) and/or preservative systems and polyacrylamide.

The medicinal clay can be treated or delivered in various forms to ensure the desired ORP, CEC, and pH of the medicinal clay is set to desired specifications, defined herein, to assure anti-pathogenic activity remains within viable ranges. In an embodiment the medicinal clay retains a Cation Exchange Capacity of at least about greater than about 10 mEq/100 g, an Oxidation-Reduction Potential greater than about 250 mV, and a pH less than about 5.0. In embodiments the coated clay may be modified by a functional ingredient and/or therapeutic agent, however the modification maintains a Cation Exchange Capacity of at least about greater than about 10 mEq/100 g, an Oxidation-Reduction Potential greater than about 250 mV, and a pH less than about 5.0.

A further step of sterilizing a medicinal clay can optionally be employed depending on the intended application. Sterilization methods should be validated to ensure effective sterilization without negatively impacting product stability or anti-pathogenic activity measures of the coated or encapsulated clay material.

The medicinal clay can be provided in various forms, including flowable powders, granules, tablets, or the like, depending upon the size of the clay that is coated and the thickness of coating agent applied thereon.

In certain embodiments, the medicinal clay can be included in the composition in an amount of from about 5 wt. % to about 70 wt. %, about 10 wt. % to about 50 wt. %, about 15 wt. % to about 35 wt. %, or any range therein.

### Additional Functional Ingredients

Additional functional ingredients can optionally be included in the antimicrobial compositions. In some embodiments few or no additional functional ingredients agents are included in the compositions.

In embodiments including an additional functional ingredient, these can include components conventionally included within pharmaceutical or therapeutic preparations, including for example, preparations for topical administration. Examples that may be mentioned are additives which are suitable for producing a desired delivery or dosage form, such as film, electro-spun materials, foam, hydrocolloid, hydrogel, gel, alginate gel, gel sheet, emulsion, suspension, paste, cream, ointment, cream, powder, tablet, capsule, transdermal therapeutic system or dressing or other delivery system impregnated with the antimicrobial composition. The functional ingredients provide desired properties and functionalities to the compositions, including a material that when combined with a therapeutic agent provides a beneficial property in a particular use or treatment. In certain embodiments, the compositions can include a solvent as an additional functional ingredient. Suitable solvents include, but are not limited to, water, glycols and glycerins, rosemary, eucalyptus, ethanol, butylene glycol, propylene glycol, propanediol, isopropyl alcohol, isoprene glycol, glycerin, Carbowax (polyethylene glycol) 200, Carbowax 400, Carbowax 600, and Carbowax 800. In addition, combinations or mixtures of these solvents may be used according to the present disclosure. In one particular embodiment, the solvent is water.

Suitable viscosity adjusting agents (i.e., thickening and thinning agents) as an additional functional ingredient for the compositions include, but are not limited to, protective colloids or non-ionic gums such as pectin, chitosan, glucans, carrageenan, hydroxyethylcellulose (e.g., Cellosize HEC QP52,000-H, manufactured by Amerchol), xanthan gum, and sclerotium gum (Amigel 1.0), as well as magnesium aluminum silicate (Veegum Ultra), silica, microcrystalline wax, beeswax, paraffin, petrolatum, and cetyl palmitate. In addition, appropriate combinations or mixtures of these viscosity adjusters may be utilized.

Suitable surfactants as an additional functional ingredient for use in the compositions include, but are not limited to, additional nonionic surfactants like Surfactant 190 (dimethicone copolyol), Polysorbate 20 (Tween 20), Polysorbate 40 (Tween 40), Polysorbate 60 (Tween 60), Polysorbate 80 (Tween 80), lauramide DEA, cocamide DEA, and cocamide MEA, amphoteric surfactants like oleyl betaine and cocamidopropyl betaine (Velvetex BK-35), and cationic surfactants like Phospholipid PTC (Cocamidopropyl phosphatidyl PG-dimonium chloride). Combinations of surfactants may also be employed.

The compositions may also include one or more additional preservatives as an additional functional ingredient. Suitable preservatives include, but are not limited to, anti-microbials such as Lincoserve BDP, Germaben II (manufactured by ICI; propylene glycol, diazolidinyl urea, methylparaben, and propylparaben), Net-DTB (Isopropyl-methyl phenol), methylparaben, propylparaben, imidazolidinyl urea, benzyl alcohol, sorbic acid, benzoic acid, sodium benzoate, dichlorobenzyl alcohol, phenoxyethanol, dehydroacetic acid, and formaldehyde, as well as physical stabilizers and anti-oxidants such as alpha-tocopherol (vitamin E), sodium ascorbate/ascorbic acid, ascorbyl palmitate and propyl gallate. In addition, combinations or mixtures of these preservatives may also be used.

Various additives, known to those skilled in the art, may also be included in the compositions including those disclosed in U.S. Pat. No. 9,095,542, which is herein incorporated by reference in its entirety. In certain embodiments, for example, it may be desirable to include one or more skin permeation enhancers as an additional functional ingredient in the formulation. Examples of suitable enhancers include, but are not limited to, ethers such as diethylene glycol monoethyl ether (available commercially as Transcutol®) and diethylene glycol monomethyl ether; surfactants such as sodium laurate, sodium lauryl sulfate, cetyltrimethylammonium bromide, benzalkonium chloride, Poloxamer (231, 182, 184, P85, P105, P338), Tween (20, 40, 60, 80), and lecithin (U.S. Pat. No. 4,783,450); alcohols such as ethanol, propanol, octanol, benzyl alcohol, and the like; polyethylene glycol and esters thereof such as polyethylene glycol monolaurate (PEGML; see, e.g., U.S. Pat. No. 4,568,343); amides and other nitrogenous compounds such as urea, dimethylacetamide (DMA), dimethylformamide (DMF), 2-pyrrolidone, 1-methyl-2-pyrrolidone, ethanolamine, and diethanolamine; terpenes; alkanones; and organic acids, particularly citric acid and succinic acid. Azone® and sulfoxides such as DMSO and C10 MSO may also be used.

Other enhancers are those lipophilic co-enhancers typically referred to as “plasticizing” enhancers, i.e., enhancers that have a molecular weight in the range of about 150 to 1000, and an aqueous solubility of less than about 1 wt. %. Lipophilic enhancers include fatty esters, fatty alcohols, and fatty ethers. Examples of specific fatty acid esters include methyl laurate, ethyl oleate, propylene glycol monolaurate, propylene glycerol dilaurate, glycerol monolaurate, glycerol monooleate, isopropyl n-decanoate, and octyldodecyl myristate. Fatty alcohols include, for example, stearyl alcohol and oleyl alcohol, while fatty ethers include compounds wherein a diol or triol, e.g., a C2-C4 alkane diol or triol, is substituted with one or two fatty ether substituents.

Additional permeation enhancers will be known to those of ordinary skill in the art of drug delivery, and/or are described in the pertinent texts and literature. See, e.g., Percutaneous Penetration Enhancers, eds. Smith et al. (CRC Press, 1995).

The compositions may also comprise one or more moisturizers as an additional functional ingredient. Suitable moisturizers for use in the formulations of the present disclosure include, but are not limited to, lactic acid and other hydroxy acids and their salts, glycerin, propylene glycol, butylene glycol, sodium PCA, Carbowax 200, Carbowax 400, and Carbowax 800. Suitable emollients for use in the formulations described herein include, but are not limited to, PPG-15 stearyl ether, lanolin alcohol, lanolin, lanolin derivatives, cholesterol, propanediol, isostearyl neopentanoate, octyl stearate, mineral oil and various other oils, such as rosemary, olive oil, argon and eucalyptus, isocetyl stearate, Ceraphyl 424 (myristyl myristate), octyl dodecanol, dimethicone (Dow Corning 200-100 cps), phenyl trimethicone (Dow Corning 556), Dow Corning 1401 (cyclomethicone and dimethiconol), and cyclomethicone (Dow Corning 344), and Miglyol 840 (manufactured by Huls; propylene glycol dicaprylate/dicaprate). In addition, appropriate combinations and mixtures of any of these moisturizing agents and emollients may be used in accordance with the present invention.

Suitable fragrances and colors as an additional functional ingredient, such as FD&C Red No. 40 and FD&C Yellow No. 5, and natural colorants (e.g., vivianite) may also be used in the formulations.

Other suitable ingredients which may be included in the compositions include, but are not limited to, abrasives, absorbents, anti-caking agents, anti-foaming agents, anti-static agents, astringents (e.g., witch hazel, alcohol, and herbal extracts such as chamomile extract), binders/excipients (e.g., starches, tragacanth, celluloses, such as cellulose ethers including methyl cellulose or hydroxypropyl methyl cellulose), buffering agents, film forming agents, conditioning agents, opacifying agents, pH adjusters (e.g., citric acid and sodium hydroxide), osmotic modifiers (e.g., marine salts, sodium chloride, and potassium chloride), and protectants. Examples of each of these ingredients, as well as examples of other suitable ingredients in product formulations, may be found in publications by The Cosmetic, Toiletry, and Fragrance Association (CTFA). See, e.g., CTFA Cosmetic Ingredient Handbook, 2^{nd} edition, eds. John A. Wenninger and G. N. McEwen, Jr. (CTFA, 1992).

The compositions may also contain irritation-mitigating additives to minimize or eliminate the possibility of skin irritation or skin damage resulting from the pharmacologically active base or other components of the composition. Suitable irritation-mitigating additives include, for example: beta glucan, glucans, alpha-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; cis-urocanic acid; capsaicin; L-sulforphane; Curcumin; and chloroquine. The irritant-mitigating additive, if present, may be incorporated into the present compositions at a concentration effective to mitigate irritation or skin damage.

The compositions may also contain additional therapeutic agents. In an embodiment, at least one additional therapeutic agent is a therapeutic cation, such as silver (Ag). The at least one additional therapeutic agent can be further incorporated into the compositions to further serve as a delivery vehicle for the medicinal clay for delivery to the target site (e.g., biofilm). In an embodiment, the additional therapeutic agent is a therapeutic cation, e.g., silver (Ag). Silver has antimicrobial activity takes place after the silver atoms are oxidized by water or wound fluid and the atoms become cationic. Once chloride binds to silver cations the silver is rendered insoluble, non-antimicrobial, and ineffective. Chloride is the main nutrient in wound fluid (i.e., exudate), and it is an anion that rapidly binds to cationic silver. In an embodiment with a therapeutic cation (e.g., silver) as a therapeutic agent delivered with a medicinal clay chelates chloride found in wound fluid before the chloride contacts the silver, prior to its contact with silver to mitigate the binding of silver and chloride.

Incorporating cationic silver with the medicinal clay as an additional therapeutic agent allows the clay to bind to anions (e.g., chloride) present in wound exudate and mitigate chelation of therapeutic cations. Such exemplary therapeutic cations can include calcium (Ca²⁺), magnesium (Mg²⁺), and potassium (K⁺), sodium (Na⁺), silver (Ag⁺), hydrogen (H⁺), aluminum (Al³⁺), iron (Fe³⁺), iron (Fe²⁺), manganese (Mn²⁺), zinc (Zn²⁺) and copper (Cu²⁺). Beneficially, the therapeutic cations survive contact with the wound exudate as a result of the medicinal clay binding to the chloride in the wound fluid.

Biofilm inhibiting coatings can also be included as additional therapeutic agents. As biofilms are present in most chronic wounds and present a significant obstacle to both wound healing and penetration of antipathogenic agents. Products that are able to disrupt biofilms and inhibit biofilm formation without harming healthy surrounding tissues are vital to adequately address pathogenic bioburden and promote wound healing can be included in the compositions described herein. Clay compositions can incorporate a variety of non-cytotoxic coatings (e.g., chitosan, poloxamers, and pectin), and allow for improved penetration of the antimicrobial reduced cations provided from the medicinal clay. In some embodiments, the therapeutic agent for disrupting and inhibiting biofilm can be included in the compositions in an amount from about 0.0001-10 wt-%, or from about 0.001-5 wt-%, or from about 0.01-1 wt-%.

Beneficially, the therapeutic agent is a non-cytotoxic component. In some embodiments, the therapeutic agent does not include cationic antimicrobials, such as quaternary ammonium salts, alkyl pyridinium salts, alkyl imidazolium salts, alkyl morpholinium salts, benzethonium salts, ethoxylated quaternary ammonium salts, or the like, as disclosed in U.S. Pat. No. 9,675,077. Such exemplary cationic antimicrobials, namely benzalkonium, benzethonium, dimethyldialkylonium, alkylpyridinium and alkyltrimethylammonium cations act in a non-discrete and cytotoxic manner as they are potent and fast-acting cytotoxins. Benzalkonium chloride is a well-known cationic antimicrobial that is both cytotoxic and known to denature proteins. Such examples of cationic antimicrobials are not included in the compositions and methods described herein as they are unwanted and detrimental to treating subjects, namely wound beds and wound tissue.

These additional components can be formulated into the antimicrobial compositions. One skilled in the art will ascertain that the final formulation will determine additional agents needed for a particular vehicle of delivery form.

According to embodiments of the disclosure, the various additional functional ingredients may be provided in an antimicrobial composition in the amount from about 0 wt-% and about 70 wt-%, from about 0 wt-% and about 60 wt-%, from about 0 wt-% and about 50 wt-%, from about 0 wt-% and about 40 wt-%, from about 0 wt-% and about 30 wt-%, from about 0 wt-% and about 25 wt-%, from about 0.1 wt-% and about 70 wt-%, from about 0.1 wt-% and about 60 wt-%, from about 0.1 wt-% and about 50 wt-%, from about 0.1 wt-% and about 40 wt-%, from about 0.1 wt-% and about 30 wt-%, from about 0.1 wt-% and about 25 wt-%, from about 1 wt-% and about 50 wt-%, or from about 1 wt-% and about 25 wt-%. In addition, without being limited according to the disclosure, all ranges recited are inclusive of the numbers defining the range and include each integer within the defined range.

### Methods of Disrupting a Biofilm

The present disclosure also provides methods of disrupting, preventing, inhibiting and/or removing a biofilm formed by a microbial population on a surface. Biofilms are caused by the release of autoinducers and virulence factors and complicated by adhesion molecules and pathogen resistance. Said disrupting, preventing, inhibiting and/or removing means that biofilms are treated (including secretions, toxins and contaminants from the site) and further biofilm reconstitution is inhibited. In some embodiments, the method comprises contacting the surface with a composition comprising at least one nonionic block EO-PO copolymer, at least one preservative system, and polyacrylamide. In certain embodiments, the composition comprises at least two or at least three nonionic block EO-PO copolymers. In some embodiments, the nonionic block EO-PO copolymer comprises poloxamer 188, poloxamer 338, and/or poloxamer 407. In some embodiments, two preservative systems, for example phenoxyethanol and octenidine, are used.

While not being limited by a method of action, the nonionic block EO-PO copolymer forms micelles which dissolve the biofilm and deliver the preservative system to the microbial population. Once delivered, the preservative system kills and/or inhibits the microbial population.

Poloxamer micelles, which are aggregates of amphiphilic molecules, disrupt the structural integrity of biofilms. The amphiphilic nature of poloxamer micelles allows them to interact with both hydrophobic and hydrophilic components of the biofilm matrix. By inserting themselves into the biofilm matrix, poloxamer micelles can disrupt the cohesive forces holding the biofilm together, leading to the dispersion or detachment of biofilm organisms. In an aspect of the methods described herein, enhanced antimicrobial activity is achieved as a result of the poloxamer micelles encapsulating or solubilizing the preservative system (e.g. octenidine) which is otherwise less effective against biofilms due to the protective matrix. The presence of poloxamer micelles improve the solubility, stability, and delivery of such octenidine to the biofilm, increasing its efficacy in killing or inhibiting the growth of microorganisms within the biofilm. In an aspect of the methods described herein, the poloxamer micelles improve the bioavailability of the preservative system (e.g. octenidine) within the biofilm matrix. The unique structure of poloxamer micelles allows them to penetrate the biofilm and transport the encapsulated octenidine to the microorganisms within the biofilm layers. This enhanced penetration overcomes the limited diffusion of octenidine through the biofilm matrix, making the ingredient more accessible to target the biofilm-associated microorganisms. In a still further aspect of the methods described herein, the poloxamer micelles aid in the removal of biofilms from surfaces through the interaction with both the biofilm matrix and the surface to which the biofilm adheres. Their surfactant properties reduce the surface tension and promote the detachment of biofilm organisms from the surface. This facilitates the cleaning process and helps in the removal of biofilm remnants.

In some embodiments, the contacting step is repeated at regular intervals until the biofilm is sufficiently reduced and/or removed. “Sufficiently reduced” as used herein refers to a reduction in biofilm and/or microbial population sufficient to effect beneficial or desired results. In some embodiments, sufficiently reduced is complete clearance of a biofilm and/or microbial population. In other embodiments, sufficiently reduced is less than complete clearance of a biofilm and/or microbial population but is nonetheless a reduction sufficient to ensure reoccurrence of biofilm formation is unlikely due to the reduction of autoinducers, virulence factors, and adhesion. In other embodiments, sufficiently reduced is a level of reduction in biofilm and/or microbial population such that complete wound healing can occur without further treatment.

In some embodiments, the regular interval is, for example, daily, twice daily, every other day, once a week, twice a week, or three times a week. Regular reapplication may last from a single day, to several days, to several months, or until the biofilm is sufficiently removed, a cure is effected, or a diminution of disease state is achieved. In some embodiments, daily or more than one application per day for at least 4 days, 5 days, 6 days, 7 days, 10 days, 14 days, 21 days, 30 days or greater are provided. One of ordinary skill may readily determine optimum application rates, application methodologies, and repetition rates. In some embodiments, it is contemplated that the composition will be applied as a single treatment. In other embodiments, the composition will be applied one to four times daily. One having ordinary skill in the art would understand that both the interval and length of treatment may be varied depending on the pathogen, wound severity, amount of biofilm, and other relevant factors. Thus, the regular interval and length of treatment may be more or less frequent than these non-limiting examples.

In some embodiments, the surface is a tissue or organ of a subject. The tissue may be a wound, including a chronic wound. In other embodiments, the tissue or organ is skin, mucosal cells, intestinal track, ear canal, nasal passages or oral cavities in need of biofilm removal.

In other embodiments, the surface is a medical device, particularly those that are implanted or within the body. Non-limiting examples of a medical device include catheters, central lines, tubing, implants, prosthetics, and the like.

In some embodiments, said contacting step comprises topical application of the composition. The composition may, for example, be incorporated into, be rubbed, sprayed, spread, misted, or poured onto the surface in need of biofilm removal. One having ordinary skill in the art would understand the contacting/application method may be varied depending on the desired outcome.

The efficacy according to the methods is effective against a broad range of pathogens, including gram positive and gram-negative bacteria. Exemplary pathogens include for example, Bacillus spp., Clostridium spp. (including C. Difficile), Chlamydia spp., Escherichia spp., Staphylococcus spp., Klebsiella spp., Enterococcus spp., Acinetobacter spp., Pseudomonas spp., Streptococcus spp., Bordetella spp., Borrelia spp., Campylobacter spp., Brucella spp., Mycobacterium spp., Salmonella spp., Staphylococcus spp., including for example Escherichia Coli, Staphylococcus aureus, methicillin-resistant Staphylococcus aureus (MRSA), Staphylococcus epidermidis, Klebsiella pneumonia including Carbapenem Resistant Klebsielle pneumonia, Enterococcus faecalis, Enterococcus hirae, Acinetobacter baumannii, Pseudomonas aeruginosa, Streptococcus pyogenes, Mycobacterium terrae, and Mycobacterium avium. In addition to pathogens it is understood that viruses, fungi, Mycobacteria, yeast and spores can also be treated by the methods disclosed herein. Additional listings of pathogens, viruses, parasites and fungi suitable for treatment are disclosed in U.S. Publication No. US2021/0046186, which is incorporated by reference in its entirety.

While not being limited by a method of action, the methods disclosed herein effectively remove biofilm because the components of the composition work together to dissolve extracellular polymeric substances of the biofilm and pathogen membranes. This dissolution allows for the delivery of the preservative system into the pathogen cytosol, causing cellular death. The nonionic block EO-PO copolymers form micelles that contain the preservative system. Due to being packaged in the micelles, the preservative system can travel deep into the biofilm to kill the biofilm-forming microbes. Beneficially, the distribution of the preservative system throughout the micelles creates a large surface area over which the antimicrobial activity can act, which provides long-term protection against any new microbial release.

In some embodiments, at least three of the nonionic block EO-PO copolymers are non-ionic and non-cytotoxic and can work synergistically to dissolve the biofilm. With the biofilm dissolved, the pathogens are left unprotected and without the benefits associated with the biofilm matrix. The loss of the biofilm matrix also releases the sequestered nutrients, such as iron and DNA. Without being limited by theory, it is believed that sequestered DNA is from other pathogens that have gained knowledge on how to combat antimicrobial attacks. Thus, the bacteria “learn” how to defend themselves and removal of the “knowledge” provided by sequestered DNA can enhance efficacy. Beneficially, this deprives the bacteria of the nutrients necessary for their survival and DNA that provides defense knowledge to the pathogens and further lends to removal of the biofilm. While not being limited by theory, it is believed that excess iron is stored in the biofilm matrix because the biofilm microbes do not require the amount of iron that a typical dividing microbe needs due to metabolic inactivity. Conventional treatment products that contain chelators like EDTA can needlessly destroy nutrients that the wound needs to perform healing functions.

Pathogens are well equipped to release toxins or expel antibiotics or antiseptics from their cytosol. They are not equipped to build new biofilm matrixes at a speed equal to the composition's ability to dissolve the extracellular polymetric structures. Pathogen death by membrane dissolution ensures minimal damage to the wound bed and its' newly forming cells.

Bacterial burden and biofilm presence can be evaluated using any method or device known in the art, including for example, point of care bacterial autofluorescence imaging, such as the MolecuLight®. Bacterial autofluorescence imaging allows for the detection and monitoring of bacteria and biofilm presence in wounds, both at the time of diagnosis and throughout healing. Imaging can be taken before and after debridement, providing practitioners with visual guidance as to remaining bacterial and biofilm burden.

In addition to the methods described herein, in embodiments employing a medicinal clay for the treatment of biofilms, the cations delivered with the medicinal clay (and/or additional therapeutic cation source) can provide various benefits. The cations, which are positively charged ions, play a significant role in biofilm communication. Biofilms are complex communities of microorganisms that form on surfaces and are encased within a matrix of extracellular polymeric substances (EPS). Communication within biofilms is crucial for coordinating various activities and behaviors, such as colonization, growth, survival, and defense. Cations, such as calcium (Ca2+), magnesium (Mg2+), and iron (Fe2+), influence biofilm communication through their interactions with EPS components and microbial cells. Cations can impact and/or overload effects on biofilm communication, including for example according to the following mechanisms:

Quorum Sensing Modulation: Quorum sensing is a cell-to-cell communication mechanism used by bacteria to coordinate gene expression in response to population density. Quorum sensing, a key communication mechanism in biofilms, is disrupted by cation overload. High cation concentrations interfere with the proper functioning of quorum sensing systems, such as by inhibiting the synthesis or release of signaling molecules (autoinducers). This disruption prevents bacteria from coordinating their activities and results in a breakdown of communication.

Cations: Influence quorum sensing by interacting with the signaling molecules involved or affecting the expression of genes associated with quorum sensing. Calcium ions have been shown to influence the expression of quorum sensing genes in various biofilm-forming bacteria. Current standard of care is to use chelation to bind cations within the biofilm. For example, most wound dressings use ethylenediaminetetraacetic acid (EDTA). Thus, the use of cationic overload to treat biofilms, the opposite from conventional treatment methodologies, represents a departure from routine methods. Bacteria control the amount of and release of iron by producing homophore and siderophore proteins which regulate iron within the biofilm environment. While not wishing to be bound by theory, it is believed that cationic overload results in the delivery of more iron to the biofilm than the bacterial proteins can successfully manage, thus eliminating this bacterial defense mechanism.

EPS Stabilization: Cations interact with negatively charged EPS components, such as polysaccharides and extracellular DNA, leading to the stabilization of the biofilm matrix. This stability affects the diffusion of signaling molecules and metabolites within the biofilm, impacting intercellular communication.

Ion Channel Regulation: Cations regulate the activity of ion channels present in bacterial cell membranes. These ion channels play a crucial role in sensing and responding to environmental signals. By modulating ion channel activity, cations influence the flow of ions across the cell membrane, altering the electrochemical gradients that are involved in signal transduction and intercellular communication.

Metallo-regulatory Systems: Some cations, such as iron, serve as essential cofactors for the function of specific regulatory proteins. These metallo-regulatory systems control the expression of genes involved in biofilm formation and communication. Iron, for instance, regulate the production of siderophores, which are molecules involved in iron acquisition and affects biofilm development.

Matrix Instability: Cation overload destabilize the biofilm matrix, which is composed of extracellular polymeric substances (EPS). The excessive presence of cations interferes with the interactions between EPS components, leading to matrix disintegration. This disrupts the physical structure of the biofilm, hampering the diffusion of signaling molecules and impairing intercellular communication.

Altered Gene Expression: Excessive cations impacts gene expression patterns within biofilms. Metallo-regulatory systems, which rely on specific cations as cofactors, are disrupted, affecting the expression of genes involved in biofilm communication. Cation overload leads to dysregulated gene expression, resulting in abnormal biofilm development and impaired communication between cells.

Cell Membrane Dysfunction: Cation overload disturbs the function of ion channels and transporters present in the bacterial cell membranes. These channels and transporters play a crucial role in signal transduction and the exchange of ions and molecules. Disruption of ion channel activity leads to imbalances in intracellular ion concentrations, impairing the ability of cells to receive and process signals, thereby affecting intercellular communication.

Impaired Microbial Interactions: Biofilms often comprise diverse microbial populations that interact with each other. Cation overload disrupts these interactions by affecting the growth and survival of specific microorganisms within the biofilm. Some microorganisms may become more dominant, while others are inhibited or killed, disrupting the intricate web of communication and cooperation within the biofilm.

### Methods of Treatment Using an Antimicrobial Composition

The present disclosure also provides methods of treating a subject with compositions of the present disclosure. In some embodiments, the method comprises administering to a tissue or organ of a subject in need of treatment a composition comprising at least one nonionic block EO-PO copolymer, at least one preservative system, and polyacrylamide, and reducing microbial populations on the subject and/or removing biofilm from the tissue or organ in need of treatment. In certain embodiments, the composition comprises at least two or at least three nonionic block EO-PO copolymers. In some embodiments, the nonionic block EO-PO copolymer comprises poloxamer 188, poloxamer 338, and/or poloxamer 407. In some embodiments, two preservative systems, for example phenoxyethanol and octenidine, are used.

While not being limited by a method of action, the nonionic block EO-PO copolymer forms micelles which dissolve the biofilm and deliver the preservative system to the microbial population. Once delivered, the preservative system kills and/or inhibits the microbial population.

In some embodiments, the treatment is administered at regular intervals until the biofilm is sufficiently reduced and/or removed and the risk of autoinducer activity, adhesions, and virulence factors have been eliminated. “Sufficiently reduced” as used herein refers to a reduction in biofilm and/or microbial population sufficient to effect beneficial or desired results. In some embodiments, sufficiently reduced is complete clearance of a biofilm and/or microbial population. In other embodiments, sufficiently reduced is less than complete clearance of a biofilm and/or microbial population but is nonetheless a reduction sufficient to ensure reoccurrence of biofilm formation is unlikely. In other embodiments, sufficiently reduced is a level of reduction in biofilm and/or microbial population such that complete wound healing can occur without further treatment.

In some embodiments, the regular interval is, for example, daily, twice daily, every other day, once a week, twice a week, or three times a week. A course of treatment may last from a single day, to several days, to several months, or until the biofilm is sufficiently removed, a cure is effected, or a diminution of disease state is achieved. In some embodiments, daily or more than one treatment per day for at least 4 days, 5 days, 6 days, 7 days, 10 days, 14 days, 21 days, 30 days or greater are provided. One of ordinary skill may readily determine optimum dosage rates, dosage methodologies, and repetition rates. In some embodiments, it is contemplated that the composition will be administered as a single treatment. In other embodiments, the composition will be administered one to four times daily. One having ordinary skill in the art would understand that both the interval and length of treatment may be varied depending on the pathogen, wound severity, amount of biofilm, and other relevant factors. Thus, the regular interval and length of treatment may be more or less frequent than these non-limiting examples.

In some embodiments, said administering step comprises topical application of the composition. The composition may, for example, be rubbed, sprayed, spread, misted, or poured onto the tissue organ in need of treatment. One having ordinary skill in the art would understand the contacting/application method may be varied depending on the tissue, organ, wound, or desired outcome.

In some embodiments, the tissue is a wound. In some embodiments, the tissue is a chronic wound. In other embodiments, the tissue or organ is skin, mucosal cells, intestinal track, ear canal, nasal passages or oral cavities in need of treatment thereof.

In some embodiments, said administering step comprises topical administration of the composition. The composition may, for example, be rubbed, sprayed, spread, misted, or poured onto the tissue, organ, or wound in need of treatment. One having ordinary skill in the art would understand the administration method may be varied depending on the desired outcome.

The efficacy according to the methods is effective against a broad range of pathogens, including gram positive and gram-negative bacteria. In some embodiments, the microbial population to be treated include for example, Bacillus spp., Clostridium spp. (including C. Difficile), Chlamydia spp., Escherichia spp., Staphylococcus spp., Klebsiella spp., Enterococcus spp., Acinetobacter spp., Pseudomonas spp., Streptococcus spp., Bordetella spp., Borrelia spp., Campylobacter spp., Brucella spp., Mycobacterium spp., Salmonella spp., Staphylococcus spp., including for example Escherichia Coli, Staphylococcus aureus, methicillin-resistant Staphylococcus aureus (MRSA), Staphylococcus epidermidis, Klebsielle pneumonia including Carbapenem Resistant Klebsielle pneumonia, Enterococcus faecalis, Enterococcus hirae, Acinetobacter baumannii, Pseudomonas aeruginosa, Streptococcus pyogenes, Mycobacterium terrae, and Mycobacterium avium. In addition to pathogens it is understood that viruses, fungi, Mycobacteria, yeast and spores can also be treated by the methods disclosed herein. Additional listings of pathogens, viruses, parasites and fungi suitable for treatment are disclosed in U.S. Publication No. US2021/0046186, which is incorporated by reference in its entirety. In certain embodiments, the viscosity of the composition increases following administration to the tissue or organ. In some embodiments, the viscosity of the composition is at least about at least about 3 times higher, at least about 10 times higher, at least about 100 times higher, or at least about 1000 times higher following administration to the tissue or organ as compared to the viscosity of the composition prior to administration. The increase in viscosity beneficially allows the composition to fill tunnels and pores that would otherwise have to be packed or else missed.

### Methods of Inhibiting Quorum Sensing in a Population of Bacteria

The present disclosure also provides methods of inhibiting and/or reducing quorum sensing in a population of bacteria. Quorum sensing is the ability of certain microbes to communicate as a collective and detect the presence of other nearby cells. The microbe's primary method of communication is through autoinducers, such as acyl-homoserine lactones in gram-negative bacteria and oligopeptides in gram-positive bacteria. As bacteria quantity increases so does the quantity of inducers, allowing the cells to detect and appropriately trigger the expression of specific genes. The expression of these genes can impact the structure of the colony, select for the growth of species, lead to the development of virulence factors such as biofilm formation, and lead to antibiotic resistance. If the microbes are separated, killed, or the population is otherwise reduced, the colony can drop below the density needed for a “quorum”, thereby halting virulence. Furthermore, since virulence factors are encapsulated within the biofilm matrix, if the matrix is degraded the virulence factors can be disabled. Thus, interrupting or inhibiting quorum sensing can aid in the removal of biofilms and prevent the reoccurrence of biofilm formation.

In some embodiments, the method of inhibiting and/or reducing quorum sensing in a population of bacteria comprises contacting the population of bacteria with a composition comprising at least one nonionic block EO-PO copolymer, at least one preservative system, and polyacrylamide, wherein the nonionic block EO-PO copolymer inhibits and/or decreases the release of autoinducers from the bacteria thereby inhibiting and/or reducing bacterial quorum sensing and/or attaching to autoinducer receptors, without activation. In certain embodiments, the composition comprises at least two or at least three nonionic block EO-PO copolymers. In some embodiments, the nonionic block EO-PO copolymer comprises poloxamer 188, poloxamer 338, and/or poloxamer 407. In some embodiments, two preservative systems, for example phenoxyethanol and octenidine, are used.

While not being limited to theory, the synergistic activity of the nonionic block EO-PO copolymers combined with the preservative system dissolve the membranes of persister bacterial cells that are dormant within the biofilm and that are activated when alarmone molecules or the HipA protein are released after an antibiotic or antiseptic attack on the bacteria. In doing so, the synergistic blend blocks ongoing infections initialed by persister bacterial cells. Moreover, the nonionic block EO-PO copolymers inhibit the release of autoinducers from the persister bacterial cells by penetrating their membranes and disabling their ability to form new quorums necessary for the reactivation of the infection.

Additionally, while not being limited to theory, the composition disrupts bacterial anchoring which is necessary for quorum sensing and the formation of biofilms. In some embodiments, at least three of the nonionic block EO-PO copolymers are non-ionic and non-cytotoxic and can work synergistically to dissolve the biofilm. With the biofilm dissolved, the pathogens are left unprotected and without the benefits associated with the biofilm matrix. The loss of the biofilm matrix also releases the sequestered DNA and nutrients, such as iron, contained within the biofilm. Beneficially, this deprives the bacteria of the “knowledge” nutrients necessary for their survival and further lends to removal of the biofilm.

For example, P. aeruginosa is a common pathogen in chronic wounds and is known to be a prolific producer of biofilm and virulent factors that kill host and innate and adaptive immune cells. While P. aeruginosa has many weapons at its disposal to use in its war within a chronic wound, one of the most virulent toxins is pyocyanin. Pyocyanin is effective broadly but its attack on macrophages is most concerning. Pyocyanin specifically attaches to macrophage cell membranes and due to its virulence, it effectively disables macrophages allowing the pathogen to enter the macrophage and sequester its iron. It is believed that the macrophages' abundance of iron makes it the key target of P. aeruginosa's pyocyanin toxic activity. The polymer blend in compositions of the present disclosure effectively block pyocyanin by dissolution, thus preserving macrophages and maintaining control of the iron sequestered within the macrophages. Without adequate iron, P. aeruginosa cannot survive and their disruptive behavior is eliminated.

Pathogens are well equipped to release toxins or expel antibiotics or antiseptics from their cytosol. They are not equipped to build new biofilm matrixes at a speed equal to the composition's ability to dissolve the extracellular polymetric structures. Pathogen death by membrane dissolution ensures minimal damage to the wound bed and its' newly forming cells. Additionally, the virulence factor alpha-toxin attacks cells by creating pores in the target cell membranes. The increased porosity causes membrane leakage and cell death. Compositions of the present disclosure coat the membranes and protect host cells from phagocytosis. At least two of the poloxamers can effectively block the activity of alpha-toxin and they can work individually or collectively in cell defense. In some embodiments, the composition inhibits and/or decreases the release of autoinducers from the bacteria thereby inhibiting bacterial quorum sensing. In some embodiments, the autoinducer is autoinducer-2 (AI-2). Without being limited by theory, AI-2 is believed to be a precursor to the development of quorum sensing and the establishment of biofilm. Thus, targeting AI-2 can aid in the elimination of established biofilm and further help prevent the further formation of biofilm.

Quorum sensing can be monitored by quantification of bacterial autoinducers, such as AI-2. Although most autoinducers are species-specific, AI-2 is produced and detected by many species of bacteria. Autoinducers such as AI-2 can be detected and quantified using methods known in the art, such as by measuring bioluminescence production and assays measuring fluorescence resonance energy transfer (FRET). See, e.g., Taga et al., Methods for Analysis of Bacterial Autoinducer-2 Production, Current Protocols in Microbiology 1C1.1-1C1.15 (November 2011).

### Methods of Treating Epidermolysis Bullosa

In certain embodiments, compositions of the present disclosure can be used to treat Epidermolysis Bullosa (EB). EB is a group of rare genetic skin disorders characterized by fragile skin that is prone to blistering and tearing from minor mechanical trauma. Currently, no cure exists for EB, leaving the only option for management to be daily wound care.

EB and third-degree burns share many similarities in terms of the skin damage they cause, but they have distinct underlying causes and pathophysiology. Both EB and third-degree burns result in severe damage to the skin. In EB, the skin is inherently fragile due to genetic mutations that affect the proteins responsible for maintaining the integrity of the skin layers. Even minor trauma or friction can cause blisters and erosions. In contrast, third-degree burns result from exposure to intense heat, chemicals, electricity, or radiation, which causes full-thickness destruction of the skin and underlying tissues. Healing in both conditions can be challenging. In EB, the fragile skin can lead to delayed wound healing, and blisters can easily rupture, leading to open sores. Similarly, third-degree burns often require specialized wound care, skin grafts, or other surgical interventions for healing. EB and third-degree burns can lead to significant scarring. In EB, repeated blistering and healing cycles can result in chronic wounds and scarring.

In certain embodiments, compositions of the present disclosure are applied topically to the patient's skin. In certain embodiments, the composition is applied via spraying so as to not cause mechanical damage to the skin. In certain embodiments, the composition is applied to the entire body of the patient, so as to cover all skin surfaces. While not wishing to be limited by theory, it is believed that compositions of the present disclosure can aid in the treatment and management of EB by forming an invisible barrier between the patient's skin and the environment. It is believed that the composition delivers cations via the combination of micelles created by the polymers. The micelles form an invisible cushioning barrier between the patient and the environment. The thin barrier reduces the risks associated with rubbing and provides an effective second skin with a cooling effect. The composition increases the comfort of the patient and additionally aids in wound healing and scar reduction.

In certain embodiments, the composition is applied daily, twice daily, or as needed. The composition does not comprise antibiotics and is non-cytotoxic, making it safe for long-term and repeated use. EB patients have devitalized skin that is in need of nutrients. Beneficially, these nutrients are provided to the patient's skin by compositions of the present disclosure, aiding in wound healing.

### EXAMPLES

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

### Example 1

Antimicrobial efficacy of the compositions described herein can be evaluated using in vitro and/or in vivo studies.

Synergy of the components can be shown through testing wherein various formulations comprising different combinations of components (according to, for example, Table 3) are compared to each other and to a control for efficacy of biofilm removal and/or microbial death. Table 3 merely presents several exemplary combinations for testing. Any combinations may be used even if not explicitly presented below.

TABLE 3
“X” means that the compound is included in the exemplary formulation.

| | 1 | 2 | 3 | 5 | 6 | 7 | 8 | 9 | 10 | 11 | 12 | 13 | 14 | 15 |
|---|---|---|---|---|---|---|---|---|---|---|---|---|---|---|
| Poloxamer 338 | X | | | X | | | X | X | | X | X | X | X | X |
| Poloxamer 188 | | X | | | X | | X | | X | X | X | X | X | X |
| Poloxamer 407 | | | X | | | X | | X | X | X | X | X | X | X |
| polyacrylamide | | | | | | | | | | | | X | | X |
| Octenidine | | | | X | X | X | X | X | X | X | X | X | | |
| phenoxyethanol | | | | | | | | | | | X | X | X | X |

### Example 2

Studies similar to those conducted in Example 1 may be conducted specifically to determine the ideal combination of poloxamers for micelle formation. Different combinations of poloxamers can be tested to determine which combinations have the most ideal features. For example, ideal combinations may result in micelles capable of encapsulating larger preservative loads. Alternatively and/or additionally, ideal combinations may result in micelles having improved biofilm dissolution rates as compared to other combinations and a control.

### Example 3

The number of micelles delivered per milliliter of composition solution can be determined. Beneficially, knowing the number of micelles delivered per milliliter aids in dosing. Providing more micelles than the microbes can overcome allows the innate immune system to reestablish and use iron released from the biofilm matrix to kill any remaining microbes. For example, In 1 mL of some embodiments of the presently disclosed composition, there are 5.18×10\hat{ }17 micelles, whereas in 1 g of Pseudomonas aeruginosa there are around 1.67×10\hat{ }5 cells, meaning there would be 3.10×10\hat{ }12 micelles per bacteria cell of Pseudomonas aeruginosa or you would have (3.22×10\hat{ }-13 Pseudomonas aeruginosa per micelle).

### Formulation 1

The number of micelles that would form in a system with 13.33% poloxamer 338, 13.33% poloxamer 407, and 13.33% poloxamer 188 with 57.65% water and at room temperature can be determined. The number of delivered micelles would depend on various factors such as the critical micelle concentration (CMC) and the interactions between the different types of poloxamers. Since each of the poloxamers are above their respective CMCs and there are no significant interactions between them, the number of micelles would depend on the total amount of poloxamer present in the system.

In this case, the total amount of poloxamer present in the system is 40% (13.33%+13.33%+13.33%). Assuming an average molecular weight of 15,050 g/mol for poloxamer 338, 12,300 g/mol for poloxamer 407, and 8440 g/mol for poloxamer 188, the total amount of poloxamer in the system would be:

$$( 0.1333 * 15050 ) + ( 0.1333 * 12300 ) + ( 0.1333 * 8440 ) = 4770.81 g$$

Assuming that each micelle contains an average of 100 poloxamer molecules, the total number of micelles in the system would be:

$$4770.81 g / ( 100 * 11916.66 g / mol ) = 4. \times 10 \hat{} ( - 3 ) mol$$

The number of micelles would also depend on the volume of the system. Assuming a total volume of 100 mL (57.65 mL water+40 mL poloxamer mixture+2.35 mL other), the concentration of the poloxamer mixture would be:

$$4770.81 g / 100 mL = 47.7081 g / mL$$

Assuming a density of 0.928 g/mL for the poloxamer mixture, the total mass of the poloxamer mixture in the system would be:

$$40 mL * \text{.928} g / mL * 47.7081 g / mL = 1770.93 g$$

The number of micelles would then be:

$$4. 10 \hat{} ( - 3 ) mol * ( 6.022 \times 10 \hat{} 23 molecules / mol ) * ( 1770.93 g / ( 11916.66 g / mol ) ) = 3.57 \times 10 \hat{} 20 micelles per 100 mL of solution or 3.57 \times 10 \hat{} 18 per 1 mL of solution$$

The total amount of octenidine-phenoxyethanol blend (OPE) in 100 mL of this exemplary formulation would be 1.2528. This means that there would be 3.51×10\hat{ }-21 g of OPE per micelle in this exemplary formulation.

### Formulation 2

The number of micelles that would form in a system with 9% poloxamer 338, 15% poloxamer 407, and 6% poloxamer 188 with 67.65% water and at room temperature can be determined. The number of delivered micelles would depend on various factors such as the critical micelle concentration (CMC) and the interactions between the different types of poloxamers. Since each of the poloxamers are above their respective CMCs and there are no significant interactions between them, the number of micelles would depend on the total amount of poloxamer present in the system.

In this case, the total amount of poloxamer present in the system is 30% (9%+15%+6%). Assuming an average molecular weight of 15,050 g/mol for poloxamer 338, 12,300 g/mol for poloxamer 407, and 8440 g/mol for poloxamer 188, the total amount of poloxamer in the system would be:

$$( 0.09 * 15050 ) + ( 0.15 * 12300 ) + ( 0.06 * 8440 ) = 3705.9 g$$

Assuming that each micelle contains an average of 100 poloxamer molecules, the total number of micelles in the system would be:

$$3705.9 g / ( 100 * 12353 g / mol ) = 3. \times 10 \hat{} ( - 3 ) mol$$

The number of micelles would also depend on the volume of the system. Assuming a total volume of 100 mL (67.65 mL water+30 mL poloxamer mixture+2.35 mL other), the concentration of the poloxamer mixture would be:

$$3705.9 g / 100 mL = 37.06 g / mL$$

Assuming a density of 928 g/mL for the poloxamer mixture, the total mass of the poloxamer mixture in the system would be:

$$30 mL * \text{.928} g / mL * 37.06 g / mL = 1031.75 g$$

The number of micelles would then be:

$$3. 10 \hat{} ( - 3 ) mol * ( 6.022 \times 10 \hat{} 23 molecules / mol ) * ( 1031.75 g / ( 12353 g / mol ) ) = 1.51 \times 10 \hat{} 20 micelles per 100 mL of solution or 1.51 \times 10 \hat{} 18 per 1 mL of solution$$

The total amount of octenidine-phenoxyethanol blend (OPE) in 100 mL of this exemplary formulation would be 1.2528. This means that there would be 8.30×10\hat{ }-21 g of OPE per micelle in this exemplary formulation.

### Formulation 3

The number of micelles that would form in a system with 15% poloxamer 338, 5% poloxamer 407, and 1% poloxamer 188 with 76.65% water and at room temperature can be determined. The number of delivered micelles would depend on various factors such as the critical micelle concentration (CMC) and the interactions between the different types of poloxamers. Since each of the poloxamers are above their respective CMCs and there are no significant interactions between them, the number of micelles would depend on the total amount of poloxamer present in the system.

In this case, the total amount of poloxamer present in the system is 21% (15%+5%+1%). Assuming an average molecular weight of 15,050 g/mol for poloxamer 338, 12,300 g/mol for poloxamer 407, and 8440 g/mol for poloxamer 188, the total amount of poloxamer in the system would be:

$$( 0.15 * 15050 ) + ( 0.05 * 12300 ) + ( 0.01 * 8440 ) = 2956.9 g$$

Assuming that each micelle contains an average of 100 poloxamer molecules, the total number of micelles in the system would be:

$$2956.9 g \text{/(} 100 * 14060 g / mol ) = 2.1 \times 10 \hat{} ( - 3 ) mol$$

The number of micelles would also depend on the volume of the system. Assuming a total volume of 100 mL (76.65 mL water+21 mL poloxamer mixture+2.35 mL other), the concentration of the poloxamer mixture would be:

$$2956.9 g / 100 mL = 29.57 g / mL$$

Assuming a density of 928 g/mL for the poloxamer mixture, the total mass of the poloxamer mixture in the system would be:

$$21 mL * \text{.928} g / mL * 29.57 g / mL = 576.26 g$$

The number of micelles would then be:

$$2.1 10 \hat{} ( - 3 ) mol * ( 6.022 \times 10 \hat{} 23 molecules / mol ) * ( 576.26 g / ( 14060 g / mol ) ) = 5.18 \times 10 \hat{} 19 micelles per 100 mL of solution or 5.18 \times 10 \hat{} 17 per 1 mL of solution$$

The total amount of octenidine-phenoxyethanol blend (OPE) in 100 mL of this exemplary formulation would be 1.2528. This means that there would be 2.42×10\hat{ }-20 g of OPE per micelle in this exemplary formulation.

### Formulation 4

The number of micelles that would form in a system with 8.57% poloxamer 338, 2.86% poloxamer 407, and 0.57% poloxamer 188 with 76.65% water can be determined in a similar way.

In this case, the total amount of poloxamer present in the system is 12% (8.57%+2.86%+0.57%). Assuming an average molecular weight of 15,050 g/mol for poloxamer 338, 12,300 g/mol for poloxamer 407, and 8440 g/mol for poloxamer 188, the total amount of poloxamer in the system would be:

$$( 0 \text{.0857} * 5050 ) + ( 0 \text{.0286} * 12300 ) + ( 0 \text{.0057} * 8440 ) = 1689.6 \; 73 g$$

Assuming that each micelle contains an average of 100 poloxamer molecules, the total number of micelles in the system would be:

$$1689 \text{.673} / ( 100 * 14060 g / mol ) = 1.2 \times 10 \hat{} ( - 3 ) mol$$

The number of micelles would also depend on the volume of the system. Assuming a total volume of 100 mL (86.65 mL water+12 mL poloxamer mixture+1.35 mL other), the concentration of the poloxamer mixture would be:

$$1689.6 \; 73 g / 100 mL = 16.8 \; 9673 g / mL$$

Assuming a density of 923 g/mL for the poloxamer mixture, the total mass of the poloxamer mixture in the system would be:

$$12 mL * \text{.923} g / mL * 16.89673 g / mL = 187.15 g$$

The number of micelles would then be:

$$1.2 10 \hat{} ( - 3 ) mol * ( 6.022 \times 10 \hat{} 23 molecules / mol ) * ( 187.15 g / ( 14060 g / mol ) ) = 9.62 \times 10 \hat{} 18 micelles per 100 mL of solution or 9.62 \times 10 \hat{} 16 per 1 mL of solution$$

The total amount of OPE in 100 mL of this exemplary formulation would be 1.2528 g. This means that there would be 1.30×10\hat{ }-19 g of OPE per micelle in this exemplary formulation.

### Formulation 5

The number of micelles that would form in a system with 1.5% poloxamer 338, 2.5% poloxamer 407, and 1% poloxamer 188 with 93.65% water and at room temperature can be determined. The number of delivered micelles would depend on various factors such as the critical micelle concentration (CMC) and the interactions between the different types of poloxamers. Since each of the poloxamers are above their respective CMCs and there are no significant interactions between them, the number of micelles would depend on the total amount of poloxamer present in the system.

In this case, the total amount of poloxamer present in the system is 5% (1.5%+2.5%+1%). Assuming an average molecular weight of 15,050 g/mol for poloxamer 338, 12,300 g/mol for poloxamer 407, and 8440 g/mol for poloxamer 188, the total amount of poloxamer in the system would be:

$$( 0.015 * 15050 ) + ( 0.025 * 12300 ) + ( 0.01 * 8440 ) = 617.65 g$$

Assuming that each micelle contains an average of 100 poloxamer molecules, the total number of micelles in the system would be:

$$617.65 g / ( 100 * 12353 g / mol ) = 500 \times 10 \hat{} ( - 4 ) mol$$

The number of micelles would also depend on the volume of the system. Assuming a total volume of 100 mL (93.65 mL water+5 mL poloxamer mixture+2.35 mL other), the concentration of the poloxamer mixture would be:

$$617.65 g / 100 mL = 6.1 \; 765 g / mL$$

Assuming a density of 923 g/mL for the poloxamer mixture, the total mass of the poloxamer mixture in the system would be:

$$5 mL * \text{.923} g / mL * 6.1765 g / mL = 28.5 g$$

The number of micelles would then be:

$$5. 10 \hat{} ( - 4 ) mol * ( 6.022 \times 10 \hat{} 23 molecules / mol ) * ( 28.5 g / ( 12353 g / mol ) ) = 6.95 \times 10 \hat{} 17 micelles per 100 mL of solution or 6.95 \times 10 \hat{} 15 per 1 mL of solution$$

The total amount of octenidine-phenoxyethanol blend (OPE) in 100 mL of this exemplary formulation would be 1.2528. This means that there would be 1.8×10\hat{ }-18 g of OPE per micelle in this exemplary formulation.

### Formulation 6

The number of micelles that would form in a system with 0.66% poloxamer 338, 0.66% poloxamer 407, and 0.66% poloxamer 188 with 96.65% water and at room temperature can be determined. The number of delivered micelles would depend on various factors such as the critical micelle concentration (CMC) and the interactions between the different types of poloxamers. Since each of the poloxamers are above their respective CMCs and there are no significant interactions between them, the number of micelles would depend on the total amount of poloxamer present in the system.

In this case, the total amount of poloxamer present in the system is 40% (0.66%+0.66%+0.66%). Assuming an average molecular weight of 15,050 g/mol for poloxamer 338, 12,300 g/mol for poloxamer 407, and 8440 g/mol for poloxamer 188, the total amount of poloxamer in the system would be:

$$( 0.0066 * 15050 ) + ( 0.0066 * 12300 ) + ( 0 \text{.0066} * 8440 ) = 236.2 \; 14 g$$

Assuming that each micelle contains an average of 100 poloxamer molecules, the total number of micelles in the system would be:

$$236.2 \; 14 g / ( 100 * 11916.66 g / mol ) = 1.98 \times 10 \hat{} ( - 4 ) mol$$

The number of micelles would also depend on the volume of the system. Assuming a total volume of 100 mL (96.65 mL water+2 mL poloxamer mixture+1.35 mL other), the concentration of the poloxamer mixture would be:

$$236.2 \; 14 g / 100 mL = 2.3 \; 6214 g / mL$$

Assuming a density of 923 g/mL for the poloxamer mixture, the total mass of the poloxamer mixture in the system would be:

$$2 mL * \text{.923} g / mL * 2.36214 g / mL = 4.36 g$$

The number of micelles would then be:

$$1.98 10 \hat{} ( - 4 ) mol * ( 6.022 \times 10 \hat{} 23 molecules / mol ) * ( 4.36 g / ( 11916.66 g / mol ) ) = 4.36 \times 10 \hat{} 16 micelles per 100 mL of solution or 4.36 \times 10 \hat{} 14 per 1 mL of solution$$

The total amount of octenidine-phenoxyethanol blend (OPE) in 100 mL of this exemplary formulation would be 1.2528. This means that there would be 2.87×10\hat{ }-18 g of OPE per micelle in this exemplary formulation.

### Example 4

The amount of preservative that can be encapsulated and delivered can also be calculated.

### Octenidine System

To calculate how much of an octenidine system can be encapsulated and delivered by the tails of a poloxamer blend with 129 PEO heads and 46 PPO tails, the weight fraction of the PPO tails in the blend must first be determined.

The total molecular weight of the poloxamer blend can be calculated as follows:

$$Total molecular weight = ( 129 \times 20.317441 ) + ( 46 \times 16.832391 ) = 2668.835$$

The weight fraction of the PPO tails can be calculated as follows:

$$Weight fraction of PPO tails = ( 46 \times 16.832391 ) / 2668.835 = 0.2904$$

Assuming that the octenidine system is similar in density to water, the formula for micelle capacity can be used to estimate how much of the octenidine system can be encapsulated:

$$Micelle capacity = ( 1.043 / ( 1 + ( Kd / Ce ) ) ) \times ( N \times MW )$$

Where Kd is the dissociation constant for the drug-polymer interaction, Ce is the drug concentration in the bulk solution, N is the number of monomers in the micelle, and MW is the molecular weight of the drug.

$$Micelle capacity = ( 1.043 / ( 1 + ( Kd / Ce ) ) ) \times ( 46 \times 16.832391 \times 10 \hat{} - 3 ) \times ( 302.45 g / mol )$$

The values for Kd or Ce are unknown. Thus, assumptions are made that the drug concentration is very low and the dissociation constant is high, meaning that all of the drug will be encapsulated. It can also be assumed that the micelle is made up of all 46 PPO tails.

Using the molecular weight of the octenidine system (MW=302.45 g/mol), we can calculate the micelle capacity as follows:

$$Micelle capacity = ( 46 \times 16.832391 \times 10 \hat{} - 3 ) \times ( 302.45 g / mol )$$

Octenidine system capacity=234.18 mg/g

Therefore, the PPO tails in the poloxamer blend with 129 PEO heads and 46 PPO tails can encapsulate and deliver approximately 234.18 mg of an octenidine system per gram of PPO tails.

### Phenoxyethanol

To calculate how much phenoxyethanol can be encapsulated and delivered by the tails of a poloxamer blend with 129 PEO heads and 46 PPO tails, we need to first determine the weight fraction of the PPO tails in the blend must first be determined.

The total molecular weight of the Poloxamer blend can be calculated as follows:

$$Total molecular weight = ( 129 \times 20.317441 ) + ( 46 \times 16.832391 ) = 2668.835$$

The weight fraction of the PPO tails can be calculated as follows:

$$Weight fraction of PPO tails = ( 46 \times 16.832391 ) / 2668.835 = 0.2904$$

Assuming that Phenoxyethanol is similar in density to water, the formula for micelle capacity can be used to estimate how much of the phenoxyethanol can be encapsulated:

$$Micelle capacity = ( 1.121 / ( 1 + Kd / Ce ) ) ) \times ( N \times MW )$$

Where Kd is the dissociation constant for the drug-polymer interaction, Ce is the drug concentration in the bulk solution, N is the number of monomers in the micelle, and MW is the molecular weight of the drug.

$$Micelle capacity = ( 1.121 / ( 1 + ( Kd / Ce ) ) ) \times ( 46 \times 16.832391 \times 10 \hat{} - 3 ) \times ( 138.16 g / mol )$$

The values for Kd or Ce are unknown. Thus, assumptions are made that the drug concentration is very low and the dissociation constant is high, meaning that all of the drug will be encapsulated. It can also be assumed that the micelle is made up of all 46 PPO tails.

Using the molecular weight of phenoxyethanol (MW=138.16 g/mol), we can calculate the micelle capacity as follows:

$$Micelle capacity = ( 46 \times 16.832391 \times 10 \hat{} - 3 ) \times ( 138.16 g / mol )$$

Phenoxyethanol Micelle capacity=106.96 mg/g

Therefore, the PPO tails in the poloxamer blend with 129 PEO heads and 46 PPO tails can encapsulate and deliver approximately 106.98 mg of phenoxyethanol per gram of PPO tails.

### Example 5

Compounds and methods of the present disclosure are effective at reducing/removing biofilm and treating chronic wounds, as illustrated by the following case studies.

### Case Study 1

An 89-year-old male patient presented with a chronic wound on his right foot that had been very slow to resolve. The patient had a medical history of type two diabetes, stage 4 renal failure, morbid obesity, peripheral arterial disease, and severe edema of the lower extremities.

Upon presentation, the patient had a large hematoma resulting from a leg injury sustained at a dental office. The objective of the treatment plan was to avoid a skin graft due to his compromised medical status. His plan involved weekly debridement and dressings changes in the office. He cared for his wound at home with a dressing change every other day. The patient was instructed to cleanse the wound with purified water and apply an antimicrobial composition of the present disclosure over the entire wound. He was then to apply a silicon dressing over the wound and treatment to protect the wound and the surrounding tissue.

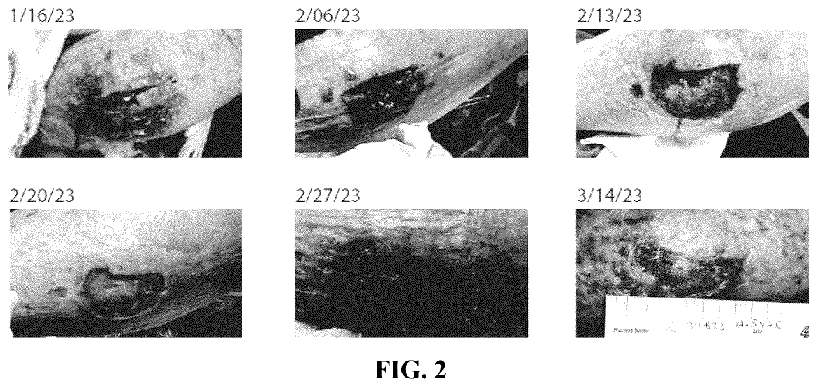

The patient's wound showed excellent results within the first couple of weeks, as shown in FIG. 2. There were no signs of infection and the treatment with the antimicrobial composition remained the only wound care product used on the patient.

### Case Study 2

A 61-year-old male with Cauda equina syndrome presented with a stage three ulcer on the right foot in the area of the anterior tibia calcaneus area that had been resistant to treatment for over two years. The patient had a medical history of polyneuropathy in the lower extremities. As an outcome of his polyneuropathy, the chronic wound on his foot developed. The patient also had a spinal injury, secondary to clot, causing ischemic spinal cord. The patient had multiple procedures in the past in an effort to treat the chronic wound, including split thickness skin graft.

The patient was treated with weekly debridement and dressing changes in the office with an antimicrobial composition of the present disclosure and a commercially available silver-based treatment for the first month and then the antimicrobial composition only after that time. Between his weekly visits, the patient did a dressing change every other day at home following the same protocol.

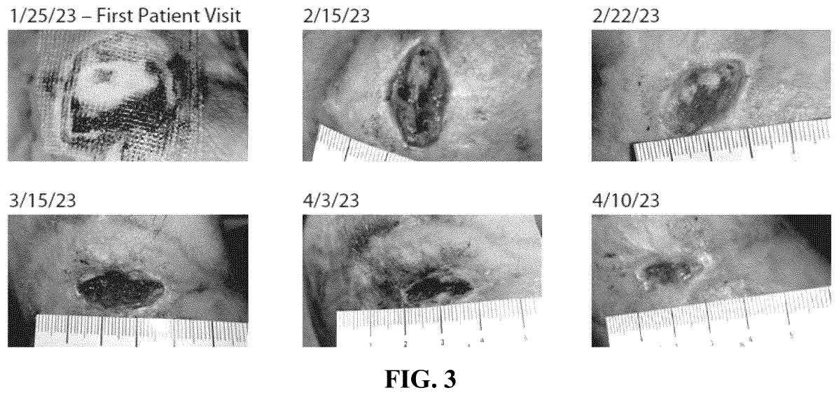

As can be seen in FIG. 3, over a period of less than three months, the chronic wound decreased dramatically in size.

### Case Study 3

A patient with a history of chronic edema presented with an edema ulcer on the right leg. The wound had failed to progress on standard wound care.

The patient was started on a protocol using compositions of the present disclosure. Wounds were cleansed with a non-ionic cleaner and baseline bacterial autofluorescence imaging (e.g., MolecuLight®) was performed. The wounds were debrided and cleansed with a non-ionic cleaner with attempts to remove bacteria, if possible. Bacterial autofluorescence imaging was repeated. Wounds were hydrated with a poloxamer wash composition of the present disclosure. Then, a thermoreversible, antimicrobial poloxamer composition of the present disclosure and a commercially available antimicrobial Fentonite™ suspension were applied. Secondary dressings and compressions, or off-loading, were applied as appropriate for specific wound. Dressings were changed twice per week with non-ionic cleanser and debridement as necessary. Bacterial autofluorescence imaging was performed before and after each debridement to monitor progress of bacteria and biofilm reduction.

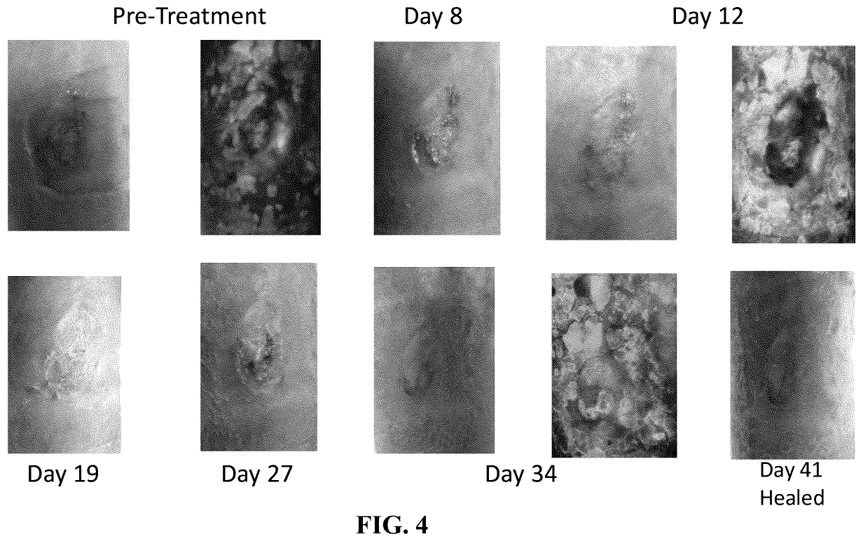

FIG. 4 shows healing progression of the wound following commencement of the treatment protocol. As can be seen, the chronic edema wound healed within 41 days of the start of treatment with significant improvement seen within the first two weeks.

### Case Study 4

A 74-year-old male presented with a six-month history of right lower extremity edema ulcers with a fragile erythematous skin surface and superficial ulcerations with exudate going through dressings. Cultures demonstrated Pseudomonas aeruginosa resistant to all oral antibiotics.

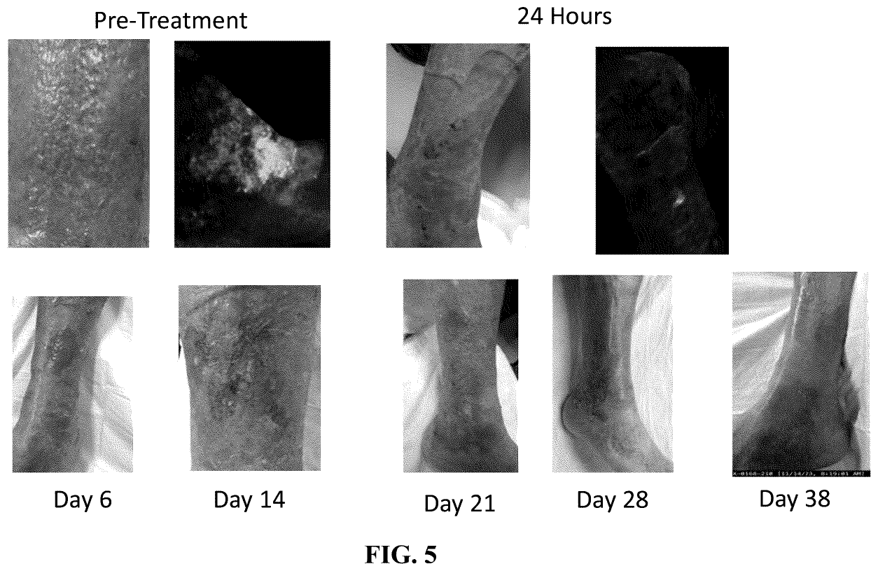

The patient began the same protocol as outlined in Case Study 3 and was seen in an outpatient clinic weekly or biweekly for assessment and replacement of compression wraps. As can be seen in FIG. 5, within 24 hours of the start of treatment, the skin surface was smooth with less erythema and improved appearance. There was a significant decrease in exudate and bacteria, as shown by the bacterial autofluorescence imaging taken after 24 hours. By day 6, there was no pain, minimal exudate, and the skin was intact. Removal of superficial dry skin demonstrated intact, healthy skin underneath. The bacterial autofluorescence imaging taken on day 14 showed dry skin but no Pseudomonas. After 38 days, the patient was transitioned from active compression to graded support stockings.

### Case Study 5

A 16-year-old male presented with long standing pilonidal disease. The patient previously had excision of the pilonidal cyst but suffered from intermittent abscess formation along the suture line. The patient was referred to a wound care clinic for treatment of the suture line complications and began the same protocol as outlined in Case Study 3.

At presentation, the distal suture line was separated, ulcerated, and had significant bacterial colonization that could not be removed with debridement and cleaning. 6 days after beginning treatment, the size of the ulcer had decreased by 50% and the bacterial burden had significantly decreased. On day 14, the incision was fully healed with no bacteria present. No further breakdown of the suture line occurred.

Pilonidal excision surgery is associated with a very high rate of suture line complications due to infection. Once the incision starts to separate and become infected, the entire suture line typically becomes involved. Thus, the ability of antimicrobial compositions of the present disclosure to fully resolve infection within two weeks is surprising and represents an improvement over traditional wound care protocols.

### Case Study 6

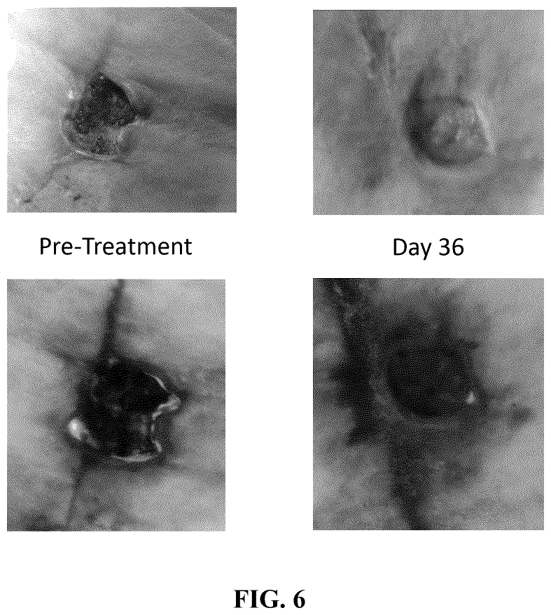

A 61-year-old male presented with a chronic wound following hip surgery that would not heal after 2 months of standard wound care. On evaluation, the wound had fibrotic borders and slough in the bottom. The wound was debrided and baseline bacterial autofluorescence imaging (e.g., MolecuLight®) was performed (FIG. 6). A similar protocol as outlined in Case Study 3 was used. As can be seen in FIG. 6, by day 36, the wound filled in from the bottom and shrank significantly in size. Furthermore, no bacteria was seen on the bacterial scan.

### Case Study 7

A 58-year-old female with alpha thalassemia presented with a left leg ulcer of 22 months following a vein harvest for popliteal bypass. The wound was previously treated with becaplermin gel and topical oxygen treatment (e.g., EO2®) for nine months with no significant change in the size of the wound.

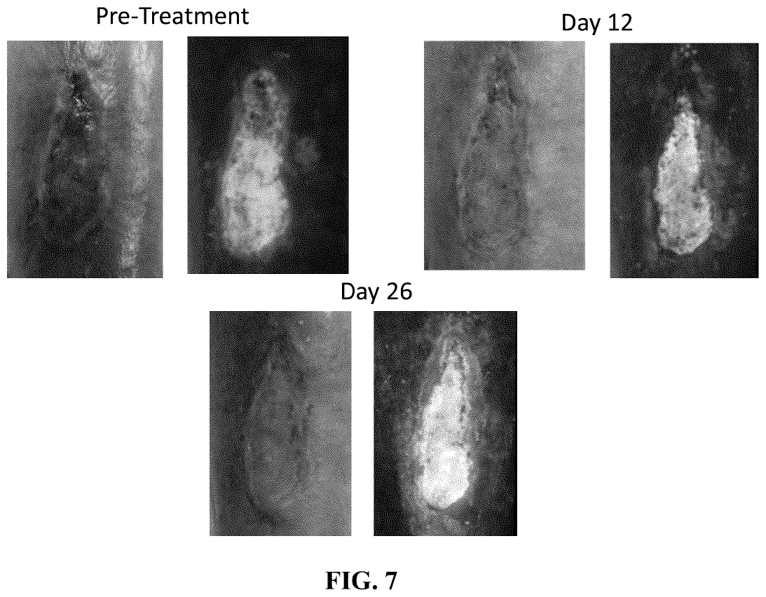

The patient was continued on becaplermin gel and topical oxygen treatment and was started on the same protocol as outlined in Case Study 3. By day 6, the patient reported a significant decrease in pain. As shown in FIG. 7, by day 12, the top of the wound was noticeably narrower with increased granulation tissue. On day 19, the patient was discontinued from the commercially available antimicrobial Fentonite™ suspension but continued on becaplermin gel, topical oxygen, and the antimicrobial poloxamer composition of the present disclosure. By day 26, the wound appearance had significantly improved and the marginal epithelium continued to increase.

These case studies demonstrate that compositions of the present disclosure are effective at curing and/or lessening the effects of chronic wounds.

### Example 6

A study was conducted to evaluate the ability of the presently disclosed compositions to prevent the formation of Pseudomonas aeruginosa.

### Materials and Methods

Three Compositions were Tested:
Test Article #1: poloxamer hydrogel
Test Article #2: commercially available antimicrobial Fentonite™ suspension
Test Article #3: poloxamer wound wash
The challenge microbial species used was P. aeruginosa (ATCC #15442)

Absorbent pads were mounted onto glass slides, the prepared slides were placed into a drip flow biofilm reactor (DFBR), and the DFBR sterilized. Sterilized polycarbonate membranes were placed on top of the absorbent pads and then the top surface of the membranes was inoculated with Pseudomonas aeruginosa. After a 30-minute air dry, a sterile rubber ring was placed over the membrane and 1 mL of the test articles dispensed inside of the ring before starting a continuous 25 mL/hour flow of dilute growth media into the DFBR at room temperature. An additional 1 mL of Test Article #1 and Test Article #3 was dispensed inside of the ring again after 24 and 48 hours of continuous flow. After 72 hours of continuous flow conditions, membranes were removed, rinsed, and transferred to containers of neutralizing eluent. Biofilm was extracted by vortexing/sonicating and extracted biofilm samples plated onto agar. Three replicates of each test article were evaluated with paired untreated control replicates. Mean log 10 and mean percent reductions attributable to each test article were calculated relative to paired untreated control replicates.

### Results and Discussion

Table 4 presents the initial concentration of the P. aeruginosa challenge suspension used for initiation of biofilm formation.

TABLE 4

| Nominal Concentration (CFU/mL) | Actual Concentration (CFU/mL) |
|---|---|
| ~1 × 10⁶ to ~1 × 10⁷ | 1.10 × 10⁸ |

Table 5 presents the microbial recoveries from the untreated control procedure and Table 6-8 present the microbial recoveries and reductions following treatment with the test compositions.

TABLE 5

| | | Average | Average Log₁₀ |
|---|---|---|---|
| | CFU/Membrane | CFU/Membrane | CFU/Membrane |
| Replicate | Recovery | Recovery | Recovery |
| 1 | 4.38 × 10⁸ | 4.57 × 10⁸ | 8.66 |
| 2 | 3.86 × 10⁸ | | |
| 3 | 5.48 × 10⁶ | | |

TABLE 6
Microbial recoveries and reduction of P. aeruginosa following
three treatments with Test Article #1 (poloxamer hydrogel).

| | | | | Mean Log₁₀ | |
|---|---|---|---|---|---|
| | | | | CFU/Membrane | Mean Percent |
| | | Average | Average Log₁₀ | Reduction Relative | Reduction Relative |
| | CFU/Membrane | CFU/Membrane | CFU/Membrane | to Untreated | to Untreated |
| Replicate | Recovery | Recovery | Recovery | Control | Control |
| 1 | <4.00 × 10¹ | <4.00 × 10¹ | <1.60 | >7.06 | >99.99% |
| 2 | <4.00 × 10¹ | | | | |
| 3 | <4.00 × 10¹ | | | | |

TABLE 7
Microbial recoveries and reduction of P. aeruginosa following three treatments with
Test Article #1 (poloxamer hydrogel) and Test Article #2 (commercially available
antimicrobial silver suspension) mixture. Inadequate rinsing techniques in replicates
1 and 2 led to lower CFU/Membrane Recovery and therefore lower average recovery.

| | | | | Mean Log₁₀ | |
|---|---|---|---|---|---|
| | | | | CFU/Membrane | Mean Percent |
| | | Average | Average Log₁₀ | Reduction Relative | Reduction Relative |
| | CFU/Membrane | CFU/Membrane | CFU/Membrane | to Untreated | to Untreated |
| Replicate | Recovery | Recovery | Recovery | Control | Control |
| 1 | 2.94 × 10⁶ | 9.81 × 10⁵ | 5.99 | 2.67 | 99.79% |
| 2 | 1.52 × 10³ | | | | |
| 3 | <4.00 × 10¹ | | | | |

TABLE 8
Microbial recoveries and reduction of P. aeruginosa following
three treatments with Test Article #3 (poloxamer wound wash).

| | | | | Mean Log₁₀ | |
|---|---|---|---|---|---|
| | | | | CFU/Membrane | Mean Percent |
| | | Average | Average Log₁₀ | Reduction Relative | Reduction Relative |
| | CFU/Membrane | CFU/Membrane | CFU/Membrane | to Untreated | to Untreated |
| Replicate | Recovery | Recovery | Recovery | Control | Control |
| 1 | <4.00 × 10¹ | <4.00 × 10¹ | <1.60 | >7.06 | >99.99% |
| 2 | <4.00 × 10¹ | | | | |
| 3 | <4.00 × 10¹ | | | | |

All three test articles exhibited more rapid antimicrobial efficacy by reducing the microbial population by 1.76 log 10 during the 15-minute neutralization evaluation exposure. Some of the observed antimicrobial efficacy during the biofilm prevention evaluation is likely attributed to immediate antimicrobial properties that reduced the microbial population before biofilm formation could begin. Future studies can be conducted to study antimicrobial efficacy following establishment of biofilm formation.

### Example 7

Additional studies were conducted to evaluate the antimicrobial activity of the presently disclosed compositions against pathogenic organisms commonly associated with skin infections and wounds.

### Materials and Methods

The organisms are prepared by inoculating the surface of Soybean-Casein Digest Agar (TSA) plates, incubated at 30 to 35° C. for 18 to 24 hours, and Sabouraud Dextrose Agar (SDA) plates, incubated at 20 to 25° C. for 3-5 days. Following the incubation period, the plates are washed with sterile Serological Saline Solution to harvest the microorganisms used and dilutions with Saline are made, plated on TSA incubated at 30 to 35° C. for 72 hours, and plated on SDA incubated at 20 to 25° C. for 3-5 days to determine the concentration. The inoculum level is then adjusted to 108 cfu/mL for use as a stock suspension. Stock suspensions are well mixed and homogenized at each inoculation interval.

The following microorganisms were used to demonstrate the antimicrobial properties of the disclosed compositions against common pathogenic organisms: Methicillin-resistant Staphylococcus aureus (ATCC 33591) (MRSA), Escherichia coli (ATCC 8739), Pseudomonas aeruginosa (ATCC 9027), Candida albicans (ATCC 10231), Klebsiella pneumoniae (ATCC 13883), Staphylococcus epidermidis (ATCC 12228), and Streptococcus pyogenes (ATCC 12344).

Positive controls are performed at initiation and completion by pour plating to enumerate inoculum levels and verify culture purity during testing and Negative controls are performed to establish sterility of media, reagents, and materials used at initiation. Neutralizer Suitability using Dey-Engley Neutralizing broth is performed with concurrently with Kill Time testing to confirm the recovery of <100 CPU of the test organism in the subculture media in the presence of product.

Duplicate 10 mL containers for each treated specimen or material concentration is prepared, equilibrated to 25±2° C., and 0.1 mL of inoculum is added to each container to achieve a final concentration of 106 cfu/mL into the product.

Serial dilutions from each replicate are made at intervals of 30 second, 1 minute, and 5 minutes using 1 ml of the inoculated test product into 9 ml Saline from 1:10 to 1:1000000. Subsequently, 1 mL from each dilution is pour plated with TSA for bacteria, incubated at 30 to 35° C. for 72 hours, and SDA for fungus incubated at 20 to 25° C. for 5 days, both in duplicate. After the incubation period, all plates are counted to determine the number of microorganisms, results are averaged and reported as log₁₀ reductions.

Results are shown in Tables 9-15. Percent reduction and log₁₀ reduction were calculated using the following formulas:

$$Percent reduction = ( initial population - surviving population ) / initial population \times 100$$

$${Log}_{10} reduction = {Log}_{10} ( initial population ) - {Log}_{10} ( surviving population )$$

TABLE 9
(MRSA) Staphylococcus aureus

| | | |
|---|---|---|
| | Concentration of Organism | |
| Exposure | cfu/mL | Percent Reduction |

| Time | Control | Product | Control | Product |
|---|---|---|---|---|
| 0 | 2.8 × 10⁶ | N/A | N/A | N/A |

| | | | | | |
|---|---|---|---|---|---|
| 1 | hour | N/A | <100 | N/A | 99.99% |
| 6 | hours | N/A | <100 | N/A | 99.99% |
| 12 | hours | N/A | <100 | N/A | 99.99% |
| 24 | hours | N/A | <100 | N/A | 99.99% |

TABLE 10
Escherichia coli

| | | |
|---|---|---|
| | Concentration of Organism | |
| Exposure | cfu/mL | Percent Reduction |

| Time | Control | Product | Control | Product |
|---|---|---|---|---|
| 0 | 2.0 × 10⁶ | N/A | N/A | N/A |

| | | | | | |
|---|---|---|---|---|---|
| 1 | hour | N/A | <100 | N/A | 99.99% |
| 6 | hours | N/A | <100 | N/A | 99.99% |
| 12 | hours | N/A | <100 | N/A | 99.99% |
| 24 | hours | N/A | <100 | N/A | 99.99% |

TABLE 11
Pseudomonas aeruginosa

| | | |
|---|---|---|
| | Concentration of Organism | |
| Exposure | cfu/mL | Percent Reduction |

| Time | Control | Product | Control | Product |
|---|---|---|---|---|
| 0 | 8.7 × 10⁶ | N/A | N/A | N/A |

| | | | | | |
|---|---|---|---|---|---|
| 1 | hour | N/A | 3.0 × 10⁴ | N/A | 99.66% |
| 6 | hours | N/A | 1.0 × 10² | N/A | 99.99% |
| 12 | hours | N/A | <100 | N/A | 99.99% |
| 24 | hours | N/A | <100 | N/A | 99.99% |

TABLE 12
Candida albicans

| | | |
|---|---|---|
| | Concentration of Organism | |
| Exposure | cfu/mL | Percent Reduction |

| Time | Control | Product | Control | Product |
|---|---|---|---|---|
| 0 | 2.0 × 10⁶ | N/A | N/A | N/A |

| | | | | | |
|---|---|---|---|---|---|
| 1 | hour | N/A | 3.2 × 10⁵ | N/A | 84.00% |
| 6 | hours | N/A | 3.0 × 10⁴ | N/A | 98.50% |
| 12 | hours | N/A | 4.0 × 10³ | N/A | 99.80% |
| 24 | hours | N/A | <100 | N/A | 99.99% |

TABLE 13
Klebsiella pneumoniae

| | | |
|---|---|---|
| | Concentration of Organism | |
| Exposure | cfu/mL | Percent Reduction |

| Time | Control | Product | Control | Product |
|---|---|---|---|---|
| 0 | 7.0 × 10⁶ | N/A | N/A | N/A |

| | | | | | |
|---|---|---|---|---|---|
| 1 | hour | N/A | 4.0 × 10² | N/A | 99.99% |
| 6 | hours | N/A | <100 | N/A | 99.99% |
| 12 | hours | N/A | <100 | N/A | 99.99% |
| 24 | hours | N/A | <100 | N/A | 99.99% |

TABLE 14
Staphylococcus epidermidis

| | | |
|---|---|---|
| | Concentration of Organism | |
| Exposure | cfu/mL | Percent Reduction |

| Time | Control | Product | Control | Product |
|---|---|---|---|---|
| 0 | 2.4 × 10⁶ | N/A | N/A | N/A |

| | | | | | |
|---|---|---|---|---|---|
| 1 | hour | N/A | <100 | N/A | 99.99% |
| 6 | hours | N/A | <100 | N/A | 99.99% |
| 12 | hours | N/A | <100 | N/A | 99.99% |
| 24 | hours | N/A | <100 | N/A | 99.99% |

TABLE 15
Streptococcus pyogenes

| | | |
|---|---|---|
| | Concentration of Organism | |
| Exposure | cfu/mL | Percent Reduction |

| Time | Control | Product | Control | Product |
|---|---|---|---|---|
| 0 | 1.0 × 10⁶ | N/A | N/A | N/A |

| | | | | | |
|---|---|---|---|---|---|
| 1 | hour | N/A | <100 | N/A | 99.99% |
| 6 | hours | N/A | <100 | N/A | 99.99% |
| 12 | hours | N/A | <100 | N/A | 99.99% |
| 24 | hours | N/A | <100 | N/A | 99.99% |

Results show that antimicrobial composition of the present disclosure result in a 99.99% reduction value in as little as 1 hour for MRSA, E. Coli, K. pneumoniae, S. epidermis, and S. pyogenes, in as little as 6 hours for P. aeruginosa, and in as little as 24 hours for (. albicans.

### Example 8

Further studies to assess concentration of cations required to block the release of autoinducers in biofilm will be conducted. As the concentration at which biofilms lose their ability to release autoinducers (signaling molecules) varies depending on the specific biofilm-forming organism, the type of autoinducer, and the environmental conditions, further studies will be conducted to measure concentrations of cations, from medicinal clays, preservative systems, and/or therapeutic cation sources, required to remove biofilm. The threshold concentration at which biofilms lose their ability to release autoinducers is influenced by multiple factors, including the sensitivity of the organism to the cations, the presence of other ions or molecules, and the overall biofilm architecture.

In general, high concentrations of cations, such as calcium (Ca²⁺) and magnesium (Mg²⁺), potentially interferes with the release and diffusion of autoinducers. These cations bind to the autoinducers, sequester them, or affect the permeability of the biofilm matrix, limiting the dispersal of signaling molecules. Consequently, the ability of biofilms to coordinate gene expression and regulate biofilm-related processes through quorum sensing may be impaired.

### Example 9

A study was conducted to evaluate the ability of an antimicrobial composition of the present disclosure to inhibit the function of the AI-2 autoinducer. The autoindicer-2 (AI-2) is a known quorum-sensing signal molecule found in many bacteria and is involved in the regulation of several processes, including biofilm formation, virulence, and antibiotic production. Detection and quantification of AI-2 was determined by using an in vitro assay, similar to the one described in Taga ME and Xavier KB, 2011, Methods for Analysis of Bacterial Autoinducer-2 Production, Current Protocols in Microbiology 1C.1.1-1C.1.15.

Briefly, the protocol employs a bioluminescent bacterial reporter strain, Vibrio harveyi BB170, which produces light in response to AI-2. Cell free culture fluids of a bacterial strain of interest are added to the reporter strain and the light production is measured using a luminometer. AI-2 activity is calculated as the induction of luminescence of BB170. V. harveyi strain BB152 (i.e., AI-2+) is used as a positive control.

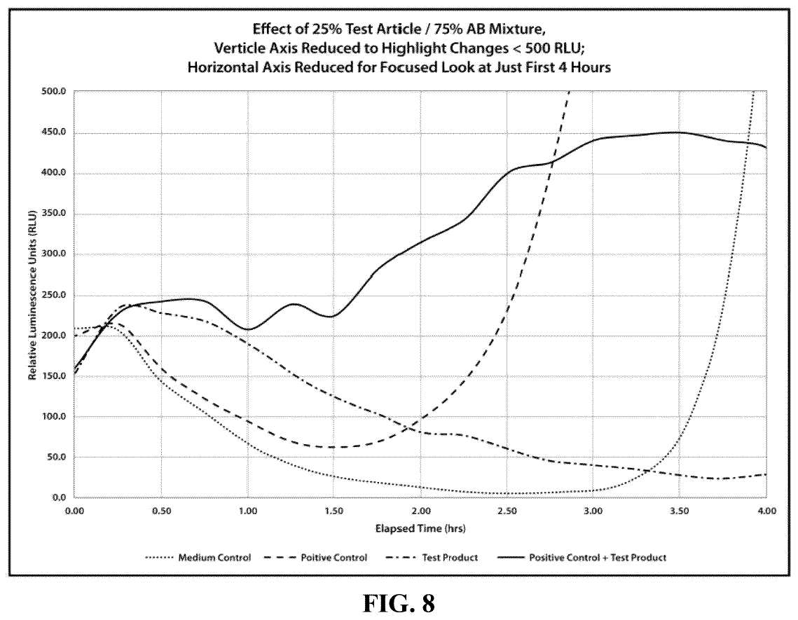

FIG. 8 shows results from this study. BB170, the reporter strain, demonstrates its functionality by producing light in response to exogenously added AI-2. This crucial observation is clearly depicted in the positive control line, where exogenous AI-2 collected from a culture of BB152 triggers a significant spike in light production. This robust response not only confirms the expected functionality of the assay but also instills confidence in the validity of the experiment.

BB170 can also produce AI-2 when a high bacterial cell density is reached and the “quorum sensing” machinery is turned on (self-induced). This phenomenon is observed on the medium control line. For the first three hours, virtually no light is produced. At around 3 hours, when the cell density has reached the necessary quorum, there is a spike in AI-2 production, represented by the rapid increase in relative luminescence units.

The positive control+test product line, presents a somewhat perplexing trend. Contrary to expectations, the luminescence gradually increased for the entire duration. This unexpected increase may be attributed to some luminescent properties of the product itself.

Of particular interest is the test product line, which represents the scenario where the test product was added to BB170 (25% test product—a 60:40 mixture of an antimicrobial clay and poloxamer hydrogel of the present disclosure/75% AB medium). It follows a similar decreasing trend, albeit with slightly higher values. However, when the cell density of BB170 reaches the threshold quorum (at about 3 hours), a distinct difference is observed. Unlike BB170 without the product (medium control line), there is no spike in AI-2 production. This suggests that products of the present disclosure inhibit AI-2 production, even when a quorum is reached.

### Example 10

Studies may be conducted to evaluate cation signaling before and after application of antimicrobial compositions of the present disclosure. Cation signaling can be measured by methods known in the art, for example, by measuring the millivoltage of the wound. The millivoltage can be measure prior to wound debridement, after debridement, and before and after dressing changes and applications of the antimicrobial composition.

It is to be understood that while the invention has been described in conjunction with the detailed description thereof, the foregoing description is intended to illustrate, and not limit the scope of the invention, which is defined by the scope of the appended claims. Other embodiments, advantages, and modifications are within the scope of the following claims. Any reference to accompanying drawings which form a part hereof, are shown, by way of illustration only. It is understood that other embodiments may be utilized and structural changes may be made without departing from the scope of the present disclosure. All publications discussed and/or referenced herein are incorporated herein in their entirety.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

1. An antimicrobial composition comprising:
at least one nonionic block EO-PO copolymer;
at least one preservative system and/or medicinal clay; and
polyacrylamide,
wherein the at least one nonionic block EO-PO copolymer encapsulates at
least a portion of the preservative system and/or medicinal clay in a micelle structure.

2. The composition of claim 1, wherein the composition comprises at least two nonionic block EO-PO copolymers.

3. The composition of claim 1, wherein the nonionic block EO-PO copolymer is a triblock polymer (PEOa-PPOb-PEOa) with the following structure 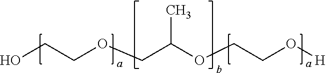
where the sum of a=70-160 and b=40-65, or a diblock polymer having an average molecular weight range of about 1,000 to about 40,000 and the weight percent content of ethylene oxide is between about 20 wt. % to about 90 wt. %.

4. The composition of claim 3, wherein the nonionic block EO-PO copolymer comprises poloxamer 188, poloxamer 338, and/or poloxamer 407.

5. The composition of claim 1, wherein the molecular weight of the nonionic EO-PO copolymer is at least 12,000 g/mol.

6. The composition of claim 1, wherein the composition comprises two preservative systems.

7. The composition of claim 1, wherein the preservative system comprises phenoxyethanol and/or octenidine.

8. The composition of claim 1, wherein the medicinal clay comprises natural clay or clay minerals, synthetic clay or clay mineral, or combinations thereof.

9. The composition of claim 8, wherein the medicinal clay comprises from about 10 to about 40 wt-% Smectite, from about 10 to about 40 wt-% Illite, and/or from about 20 to about 50 wt-% Illite-Smectite.

10-11. (canceled)

12. The composition of claim 1, wherein the polyacrylamide is crosslinked with N,N-methylenebisacrylamide.

13-15. (canceled)

16. The composition of claim 1, wherein the composition comprises from about 5 wt. % to about 30 wt. % of the nonionic block EO-PO copolymer, from about 0.05 wt. % to about 2 wt. % of the at least one preservative and/or about 10 wt. % to about 50 wt. % of the medicinal clay; and from about 0.1 wt. % to about 3 wt. % of the polyacrylamide.

17. The composition of claim 1, wherein the composition is a hydrogel, gel, alginate gel, gel sheet, emulsion, suspension, paste, cream, or ointment.

18. The composition of claim 1, wherein the viscosity of the composition is at least about 3 times higher at about 37° C. than the viscosity at about 20° C.

19. A method of disrupting a biofilm formed by a microbial population on a surface, comprising:
contacting the surface with a composition according to claim 1,
wherein the nonionic block EO-PO copolymer micelles dissolve the biofilm and delivers the preservative system and/or medicinal clay contained therein to the microbial population, and
wherein the preservative system and/or medicinal clay kills and/or inhibits the microbial population.

20. The method claim 19, wherein the said contacting step is repeated at a regular interval until the biofilm is sufficiently reduced and/or removed.

21. (canceled)

22. The method of claim 19, wherein the surface is a tissue or organ of a subject, a wound, or a medical device.

23-24. (canceled)

25. A method of treatment of a subject with a composition comprising:
administering to a tissue or organ of a subject in need of treatment a composition according to claim 1, and
reducing microbial populations on the subject and/or removing biofilm from the tissue or organ in need of treatment.

26. The method of claim 25, wherein the tissue is a wound, optionally wherein the wound is a chronic wound.

27. (canceled)

28. The method of claim 25, wherein said administering comprises topical administration.

29. (canceled)

30. The method of claim 25, wherein the microbial population comprises a population of Bacillus spp., Clostridium spp. Chlamydia spp., Escherichia spp., Staphylococcus spp., Klebsiella spp., Enterococcus spp., Acinetobacter spp., Pseudomonas spp., Streptococcus spp., Bordetella spp., Borrelia spp., Campylobacter spp., Brucella spp., Mycobacterium spp., Salmonella spp., and/or Staphylococcus spp.

31. The method of claim 30, wherein the microbial population comprises a population of methicillin-resistant Staphylococcus aureus (MRSA).

32-36. (canceled)
